(19) United States
(12) Patent Application Publication
(10) Pub. No.: US 2007/0020608 A1
(43) Pub. Date: Jan. 25, 2007

(54) Method for the generation of neural progenitor cells

(76) Inventors: Peter Eriksson, Gothenburg (SE); Mathilda Zetterstrom, Billdal (SE); Sven Enerback, Gothenburg (SE); Eva Karin Kilmare, Gothenburg (SE)

Correspondence Address: BUCHANAN, INGERSOLL & ROONEY PC, POST OFFICE BOX 1404, ALEXANDRIA, VA, 22313-1404, US

(21) Appl. No.: 10/555,306

(22) PCT Filed: May 10, 2004
(86) PCT No.: PCT/EP04/05034

§ 371 Date: Feb. 14, 2006

Related U.S. Application Data
(60) Provisional application No. 60/469,320, filed on May 8, 2003.

(30) Foreign Application Priority Data
May 8, 2003 (DK) PA 2003 00700
Jun. 27, 2003 (DK) PA 2003 00985
Jun. 27, 2003 (DK) PA 2003 00983

(51) Int. Cl. C12Q 1/00; C12N 5/08; C12N 5/06

(52) U.S. Cl. 435/4; 435/325; 435/368

(57) ABSTRACT

A rapid, simple and efficient method for the generation of neural progenitor cells from pluripotent/undifferentiated human blastocyst-derived stem (hBS) cells, neural progenitor cells obtained by the method and further differentiation of these cells into the three neural cell lineages, and the use of the neural progenitor cells and the differentiated cells in the preparation of medicaments. An important feature of the method is that neural progenitor cells are produced without a step involving formation of embryoid bodies (EB), improving the efficiency and the reducing the time for generation as compared to known methods.

### FIELD OF THE INVENTION

The present invention concerns a rapid, simple and efficient method for the generation of neural progenitor cells from pluripotent/undifferentiated human blastocyst-derived stem (hBS) cells, neural progenitor cells obtained by the method and further differentiation of these cells into the three neural cell lineages, and the use of the neural progenitor cells and the differentiated cells in the preparation of medicaments.

### BACKGROUND OF THE INVENTION

A stem cell is a cell type that has a unique capacity to renew itself and to give rise to specialized or differentiated cells. Although most cells of the body, such as heart cells or skin cells, are committed to conduct a specific function, a stem cell is uncommitted, until, it receives a signal to develop into a specialized cell type. What makes the stem cells unique is their proliferative capacity, combined with their ability to become specialized. For years, researchers have focused on finding ways to use stem cells to replace cells and tissues that are damaged or diseased. So far, most research has focused on two types of stem cells, embryonic and somatic stem cells. Embryonic stem cells are derived from the pre-implanted fertilized oocyte, i.e. blastocyst, whereas the somatic stem cells are present in the adult organism, e.g. within the bone marrow, epidermis and intestine. Pluripotency tests have shown that whereas the embryonic or blastocyst-derived stem cells (hereafter referred to as blastocyst-derived stem cells or BS cells) can give rise to all cells in the organism, including the germ cells, somatic stem cells have a more limited repertoire in descendent cell types. According to many national laws in Europe and other countries, a fertilized oocyte is not regarded as an embryo before implantation in the uterus i.e. 10-14 days after fertilization, and such cells are therefore referred to as blastocyst-derived stem cells or hBS cells herein when employed according to the invention.

Perhaps the most far-reaching potential application of hBS cells is the generation of cells and tissue that could be used for so-called cell therapies. Many diseases and disorders result from disruption of cellular function or destruction of tissues of the body. Today, donated organs and tissues are often used to replace ailing or destroyed tissue. Unfortunately, the number of people suffering from disorders suitable for treatment by these methods far outstrips the number of organs available for transplantation. The availability of hBS cells and the intense research on developing efficient methods for guiding these cells towards different cell fates, e.g. neuronal cells, holds growing promise for future applications in cell-based treatment of degenerative diseases, such as Alzheimer's and Parkinson's.

Potential applications of hBS cells themselves and cell populations derived there from are found e. g. in the drug discovery process in the pharmaceutical industry and in toxicity testings of all kinds of chemicals. Today, large-scale and high throughput screening of drug candidates usually relies on biochemical assays that provide information on compound binding affinity and specificity, but little or no information on function. Functional screening relies upon cell-based screens and usually uses organisms of poor clinical relevance such as bacteria or yeasts that can be produced cheaply and quickly at high volume. Successive rounds of screening use model species of greater clinical relevance, but these are more costly and the screening process is time consuming. Screening tools based on human primary cells or immortalised cell types exist, but these cells are limited in supply or usefulness due to loss of vital functions as a result of in vitro culture and transformation. The access to undifferentiated hBS cells and hBS cells differentiated under engineered conditions provides a new and unique capability to conduct human cell-based assays with high capacity, but without compromising clinical relevance.

Today, the methods for generation of neural progenitor cells from hBS cells, which may be further differentiated into the three neural cell lineages (astrocytes, oligodendrocytes and neurons), all include the formation of embryoid bodies and/or neurosphere formation and/or mechanical selection (Reubinoff, B. E. et al. Nat. Biotechnol. 19, 1134-1140 (2001); U.S. patent application Ser. No. 2002/0164308, Zhang, S. C. et al. Nat. Biotechnol. 19, 1129-1133 (2001), Carpenter, M. K. et al. Exp. Neurol. 172, 383-397 (2001); U.S. patent application Ser. Nos. 2002/0039724 and 2002/0009743, and International patent application published as WO 01/83715). These selection steps, especially formation of embryoid bodies, result in a major cell number loss, resulting in a low efficiency. The methods are complicated, most have very long generation times and involve several time consuming steps. Thus, there is a need for a rapid and simple method for the formation of neural progenitor cells derived from undifferentiated hBS cells.

### DESCRIPTION OF THE INVENTION

The method of the present invention relates to a rapid, very simple and fairly efficient way of generating proliferating neural progenitor cells from undifferentiated hBS cells. The method described here does not involve any formation of embryoid bodies or mechanical selection steps. This method is easy to use and cost effective compared to many other methods. It is very efficient since very little cells are lost in the generation step. The progenitors are easy to handle and proliferate well, generating large numbers of cells for further experiments. The progenitors can be easily frozen and thawed according to conventional cryopreservation techniques (Freshney RI. Instability, validation and preservation: in Culture of Animal Cells; A Manual of Basic Technique. Wiley-Liss Inc. 1994;255-265), as described in example 1.

Accordingly, the present inventors have found a rapid, simple and efficient method for the generation of neural progenitor cells from pluripotent human blastocyst-derived stem cells or cell lines.

Generation of Neural Progenitors

Thus, the present invention relates to a method for obtaining neural progenitor cells, the method comprising the steps of
i) dissociation of undifferentiated hBS cells by enzymatic and/or by mechanical treatment to obtain hBS cell aggregates or a mixture of hBS cell aggregates and single cells,
ii) seeding the hBS cell suspension from step i) in a growth medium on a support substrate,
iii) culturing the hBS cells seeded as in step ii),
iv) optionally, replacing a part of the growth medium with a growth medium,
v) passaging the obtained cells from step iii) or iv) to obtain neural progenitor cells.
vi) optionally, seeding the cells from step v) on a support substrate in a growth medium, (these cells can then also be passaged according to step v).),
vii) optionally, repeating step v) and/or vi) at least 1 time such as, e.g., from 1 to 100 times, from 1 to 75 times, from 1 to 50 times, from 1 to 40 times, from 1 to 30 times, from 1 to 20 times, and
viii) optionally, the progenitors can be frozen and thawed according to conventional cryopreservation methods and further propagated as in step v), vi) and/or vii).

An important feature of the present method is that neural progenitor cells are produced without a step involving formation of embryoid bodies (EB), improving the efficiency and the reducing the time for generation as compared to known methods.

Before going into further details of the invention, in the following is given a list of specific terms used in the present text.

Definitions and Abbreviations

As used herein, the term “blastocyst-derived stem cell” is denoted BS cell, and the human form is termed “hBS cells”.

As used herein, the term “embryoid bodies” is a term that is well-defined within the field of stem cells.

As used herein, the term “EF cells” means “embryonic fibroblast feeder”. These cells could be derived from any mammal, such as mouse or human.

By the terms “feeder cells” or “feeders” are intended to mean cells of one type that are co-cultured with cells of another type, to provide an environment in which the cells of the second type can grow. The feeder cells may optionally be from a different species as the cells they are supporting. The feeder cells may typically be inactivated when being co-cultured with other cells by irradiation or treatment with an anti-mitotic agent such as mitomycin c, to prevent them from outgrowing the cells they are supporting.

By the terms “feeder cell free” or “feeder free” is intended to mean cultures or cell populations wherein less than 5% of the total cells in the culture are feeder cells, such as, e.g., less than 4%, less than 3%, less than 2%, less than 1%, less than 0.5%, less than 0.1% and less than 0.01%. It will be recognized that if a previous culture containing feeder cells is used as a source of hBS for the culture to which fresh feeders are not added, there will be some feeder cells that survive the passage. However, after the passage the feeder cells will not grow, and only a small proportion will be viable by the end of 6 days of culture.

The starting material in step i) is pluripotent/undifferentiated hBS cells, especially hBS cells. These BS cells can be obtained from a BS cell line, especially a hBS cell line. Althogh the present invention concerns hBS cells it is contemplated that a method according to the invention also can be applicable to other mammalian BS cells.

In one embodiment of the invention the hBS cell line is established according to the method for establishing a hBS cell line described in the experimental section herein as “Establishment method”.

The hBS cells may be propagated either on feeder cells or in a feeder-free culture system prior to step i).

In one embodiment of the invention the hBS cells are propagated in a feeder-free culture system prior to step i), according to the feeder-free culture system described herein.

In another embodiment of the invention, the hBS cells have at least one of the following properties
i) exhibit proliferation capacity in an undifferentiated state for more than 12 months when grown on mitotically inactivated embryonic feeder cells or under feeder free growth conditions,
ii) exhibit normal euploid chromosomal karyotype,
iii) maintain potential to develop into derivatives of all types of germ layers both in vitro and in vivo,
iv) exhibit at least two of the following markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2,
v) do not exhibit molecular marker SSEA-1 or other differentiation markers, and
vi) retain their pluripotency and form teratomas in vivo when injected into immuno-compromised mice,
vii) are capable of differentiate.

In one embodiment the hBS cells have all the properties mentioned above.

For the generation of neural progenitors, the size of the cell aggregates obtained by the dissociation performed in step i) of the method according to the present invention affects the cells's ability to differentiate and survive in the subsequent steps. If the cell aggregates are too big, they will more readily stay as undifferentiated colonies for a longer time than smaller aggregates, when inducing neural progenitor formation. On the other hand, a pure suspension of single cells may show decreased survival rate after plating in step vii.

In one embodiment of the invention, the dissociation of hBS cells is being performed by enzymatic treatment. The enzyme used may be a collagenase, such as, e.g. collagenase type IV. The enzymatic treatment may be performed by adding 200 u/mi collagenase to the hBS cells followed by incubation at 37° C. from about 5 minutes to about 40 minutes, such as, e.g. from about 10 minutes to about 35 minutes, from about 15 minutes to about 30 minutes. The size of the cell aggregates obtained by this method may be from about 5 cells to about 200 cells, such as, e.g. from about 10 cells to about 150 cells, from about 10 cells to about 100 cells, from about 20 cells to about 100 cells, from about 30 cells to about 80 cells, from about 40 cells to about 60 cells.

After the dissociation, the hBS cells or cell aggregates are plated (seeded) onto a support substrate.

In a specific embodiment of the invention the number of cells plated onto the support substrate may be from about 40 000 cell/cm² to about 200 000 cell/cm², such as, e.g. from about 50 000 cell/cm² to about 150 000 cell/cm², from about 60 000 cell/cm² to about 100 000, from about 70 000 cell/cm² to about 80 000.

As mentioned above, the composition of the support substrate may have influence on the differentiation of the cells. At the present stage, a support substrate should be used, which favours cell adhesion and the generation of neural progenitor cells. The support substrate used in the different steps of the invention can be the same or different. Appropriate support substrates may comprise an extracellular matrix component.

Other examples of suitable support substrates, which may favour the generation of neural progenitor cells and cell adhesion are support substrates comprising one or more substances selected from the group consisting of gelatin, laminin, polyornithine, fibronectin, Matrigel™, agarose, poly-L-lysine, poly-D-lysine and collagen type I.

In a specific method according to the invention, the support substrate comprises gelatin, such as, e.g. gelatin of Type A from porcine skin (Sigma). The concentration of gelatin should be between 0.001 and 0.2%.

In a specific embodiment of the invention, the support substrate comprises gelatin, such as, e.g. gelatin of Type A from porcine skin. The concentrations of gelatin may be from about 0.001% (w/v) to about 0.2% (w/v), such as, e.g., from about 0.001% (w/v) to about 0.2% (w/v), from about 0.005% (w/v) to about 0.18% (w/v), from about 0.01% (w/v) to about 0.16% (w/v), from about 0.015% (w/v) to about 0.14% (w/v), from about 0.02% (w/v) to about 0.12% (w/v), from about 0.025% (w/v) to about 0.1% (w/v). The cell culture container may be coated with the gelatin solution for at least 10 minutes, such as, e.g., at least 15 minutes, at least 20 minutes, at least 25 minutes, at least 30 minutes at room temperature. A suitable cell culture container may be e.g. Primaria plastic plates or flasks (Falcon).

Growth Medium

Growth medias used for the generation of neural progenitor cells from hBS cells may be any suitable growth medium, such as, e.g. hBS cell medium, VitroHES™-medium or neural cell medium, Neurobasal™ media and DMEM/F12 based medias. The growth medium used in the different step of a method of the invention may be the same or different. All of these media may be used as conditioned media, such as, e.g. k-hBS medium, k-VitroHES™-medium, especially in step ii).

Conditioned media are produced by culturing a first population of cells in a medium, and then harvesting the conditioned medium by e.g. filtering. The first population of cells may be cells normally used as feeder cells, such as, e.g., mouse embryonic fibroblasts. Other suitable cells for producing conditioned medium may be adult rat hippocampal neural progenitors or cultured cells from various brain regions.

The conditioned medium (along with anything secreted into the medium by the cells) may then be used to support the growth of a second population of cells. A suitable medium for use according to the invention, is the “k-VitroHES™-medium” or “k-BS-medium”, where a monolayer of mouse and human embryonic fibroblasts is treated with mitomycin C or irradiated and then incubated with “VitroHES™-medium” or “BS-Medium” for 24 hours. The k-VitroHES™-medium or “k-BS-medium” may then be collected every day up to 3-7 times for mouse feeder and up to 3-7 times for human feeder from the same cells and sterile filtered to obtain the conditioned k-VitroHES™-medium or “k-BS-medium”. The “k-VitroHES™-medium” and “k-BS-medium” may subsequently be stored by freezing at about −20° C. or colder.

The preferred media used in the invention is VitroHES™-medium (Vitrolife, Gothenburg, Sweden) supplemented with 4 ng/ml human recombinant bFGF (basic fibroblast growth factor) or alternatively a medium termed “BS-medium” which may be comprised of; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (all from Invitrogen AB, Stockholm, Sweden).

A so-called neural cell medium, which is also suitable for use in the method of the present invention, is composed of Dulbecco's minimal essential medium (DMEM)/F12 (1:1) supplemented with N2-supplement (1:100), L-glutamine (2 mM), penicillin/streptomycin (100 units/mL; Gibco, Gaithersburg, Md.), bFGF (20 ng/mL) and EGF (20 ng/mL). All products from Invitrogen AB, Stockholm, Sweden.

Other suitable growth medias could be a media composed of Neurobasal™ media or DMEM/F12 media supplemented with B27 supplement (1:50-1:100) or N2 supplement (1:50-1:100), L-glutamine (2 mM), penicillin/streptomycin (100 units/mL; Gibco, Gaithersburg, Md.), FGF-2 (10-40 ng/mL) and/or EGF (10-40 ng/mL). All products from Invitrogen AB, Stockholm, Sweden.

Growth Factors

Growth media for use in steps ii)-viii) in the method of the present invention may comprise one or more growth factors. The growth factors used may be any suitable growth factors for the generation of neural progenitor cells. The concentration of the specific growth factor used may be important for whether the cells will differentiate further or remain in the progenitor state. The exact concentration of growth factor to be used will depend on the growth factor and the cell types.

A specific example of a growth factor usable for promoting the generation and propagation of neural progenitors is FGF, such as, e.g. FGF-2 or bFGF. The amount of FGF to be used to promote generation and propagation of neural progenitors may be from about 1 ng/ml to about 40 ng/ml, such as, e.g. from about 2 ng/ml to about 20 ng/ml, from about 3 ng/ml to about 20 ng/ml, from about 4 ng/ml to about 20 ng/ml, from about 4 ng/ml to about 15 ng/ml, from about 4 ng/ml to about 10 ng/ml or from about 4 to about 8 ng/ml.

The present inventors have found that supplementation of the growth medium with the growth factors FGF and/or epidermal growth factor (EGF), both growth factors that are known to be effective for the propagation of human fetal- and adult-derived neuroepithelial progenitors, will facilitate sequential propagation and expansion of progenitor cells.

Other suitable growth factors that promote the generation, propagation and expansion of the neural progenitor cells may of course be used.

The culturing in step iii) is performed for about 6 to 10 days, such as, e.g. for about 7 days, for about 8 days, for about 9 days or for about 10 days, before the first passage of the cells. In a specific embodiment of the invention, culturing in step iii) is performed for no longer than 8 days.

Optionally, step iv) is included, whereby part of the growth medium is replaced between the initial seeding of the hBS cells in step ii) and first passage in step v). The growth medium to be replaced and the growth medium used for replacing in step iv) can be the same or different.

In a specific embodiment of the invention, step iv) is performed after at least 1 day of culture in step iii), such as, e.g., 2 days of culture in step iii), 3 days of culture in step iii), 4 days of culture in step iii), 5 days of culture in step iii), 6 days of culture in step iii), 7 days of culture in step iii), 8 days of culture in step iii), 9 days of culture in step iii). The part of growth medium replaced in step iv) may be from about 0% (v/v) to about 100% (v/v), such as, e.g., from about 20% (v/v) to about 80% (v/v), from about 30% (v/v) to about 70% (v/v), from about 40% (v/v) to about 60% (v/v), from about 45% (v/v) to about 55% (v/v).

In step ii), iii) and iv) the cells grow in adherent three-dimensional neuraosphere-like colonies.

After the first passage in step v) the obtained cells grow as a monolayer culture.

In a specific embodiment of the invention, the cells obtained in step v) and/or vi) are further propagated by inclusion of step vii). The obtained cells may be passaged as in step vii) and viii) every 3-6 days, such as, e.g., every 4 days or every 5 days or at about 60-90% confluence, such as, e.g., at about 65-85% confluence, at about 70-80% confluence. If the progenitors are cultured for to long before passage, they will become too confluent, and there is an increasing probability of contact inhibition and/or differentiation. The optimal culturing time before passage is dependent on e.g. the seeding density of the cells and the doubling time of the cells. If the progenitors are seeded at too low densities there is an increased probability that the cells undesirably will differentiate into mature cell types. Step v) and vi) may be repeated as in step vii) from about 1 to about 30 times, such as, e.g., from about 5 to about 25 times, from about 10 to about 20 times.
b) capable of differentiating,
c) capable of self renewal.

In another embodiment of the invention, the majority of the cells obtained from step iii) and/or step iv) and/or step v) and/or step vi), and/or step vii); and/or step viii), do not exhibit one or more markers for undifferentiated hBS cells, such as, e.g., SSEA-3, SSEA-4, GCTM-2, Tra-1-60, Tra-1-80, Oct-4, Cripto, Rex-1 or FGF4.

In the present context, the term “majority of cells” is intended to denote at least about 80% of the cells such as, e.g., at least about 85%, at least about 90% or at least about 95% of the cells.

It may also be determined whether the cells express markers for cell types of other germ layers than ectoderm (neural), such as endoderm and mesoderm. For this purpose alfafetoprotein and HNF3-α (markers for endoderm) as well as cardiac troponin I, Brachyury and Desmin (markers for mesoderm) may be used.

In one embodiment of the invention, the majority of the cells obtained from step iii) and/or step iv) and/or step v) and/or step vi), and/or step vii), and/or step viii), do not exhibit markers for the endoderm germ layer, such as, e.g., alfa-fetoprotein or HNF3-α.

In another embodiment of the invention, the majority of the cells obtained from step iii) and/or step iv) and/or step v) and/or step vi), and/or step vii), and/or step viii), do not exhibit markers for the mesoderm germ layer, such as, e.g., cardiac troponin I, Brachyury or Desmin.

In order to determine whether the cells are neural progenitors cells, and not further differentiated into mature neuronal or glial cells, markers for mature neurons may be used either by RT-PCR or Immunocytochemistry analysis, such as, e.g. β-III-Tubulin, MAP2, NF-L, NF-H, NF200, NF68, DoubleCortin, ChAT, DDC, GABA, DBH, NSE, synaptophysin, TH, Nurr-1, NeuN, glutamate and serotonin. Marker for glial cell types, such as, e.g. GFAP, S-100, GaIC, RIP may also be used.

In one embodiment of the invention, the majority of the cells obtained from step iii) and/or step iv) and/or step v) and/or step vi) and/or step vii), and/or step viii), do not exhibit one or more markers for mature neuronal cells, such as, e.g., β-III-Tubulin, MAP2, NF-L, NF-H, NF200, NF68, DoubleCortin, ChAT, DDC, GABA, DBH, NSE, synaptophysin, TH, Nurr-1, NeuN, glutamate or serotonin.

In another embodiment of the invention, the majority of the cells obtained from step iii) and/or step iv) and/or step v) and/or step vi) and/or step vii), and/or step viii), do not exhibit one or more markers for glial cells, such as, e.g., GFAP, S-100, GaIC, RIP.

The cells obtained from step iii) and/or step iv) and/or step v) and/or step vi) and/or step vii), and/or step viii), may be further differentiated into at least one of the three neural cell lineages, e.g., astrocytes, oligodendrocytes, and neurons.

In one embodiment of the invention, the differentiated cells may display the expression of at least one of the following neuronal cell type markers, including neuron-specific β-III tubulin (TUJ1), NeuN, DoubleCortin, tyrosine hydroxylase, Map 2, NF-L, NH-H, NSE, DBH, GABA, synaptophysin, glutamate serotonin, Nurr-1, NF200, or NF68, or at least one of the oligodendrocyte cell type markers RIP or GaIC or at least one of the astrocyte cell type markers GFAP or S-100.

Other Aspects of the Invention

In other aspects the present invention relates to the use of neural progenitor cells obtained by the method described herein such as e.g. use in medicine and more specifically for the prevention and/or treatment of pathologies or diseases caused by tissue degeneration, such as, e.g., the degeneration of neural tissue or for the prevention or treatment of pathologies and/or diseases in the nervous system. Examples of diseases, which may be prevented and/or treated by a medicament comprising neural progenitor cells may be selected from the group consisting of multiple schlerosis, spinal chord injury, encephalopathies, Parkinson's disease, Alzheimer's disease, Huntingdon's disease, stroke, traumatic brain injuries, hypoxia induced brain injuries, ischemia induced brain injuries, hypoglycemic brain injuries, degenerative disorders of the nervous system, brain tumors and neuropathies in the peripheral nervous system.

In still other aspects, the invention relates to the use of obtained neural progenitor cells are used in an in vitro model for studying early neurogenesis or in an in vitro model for human neurodegenerative disorders. Furthermore, the invention relates to use of obtained neural progenitor cells for drug discovery, for screenings etc. and for neuro toxicity testing in vitro.

The invention also relates to a composition of neural progenitor cells (such as an essentially pure composition) obtained by a method as described herein. In such a composition the cells obtained may exhibit at least one of the neural progenitor cell type markers selected from the group consisting of nestin, internextin, PSA-NCAM (or PS-NCAM or NCAM), Musashi-1, GAP43, Cystatin C, Vimentin, A2B5, AC133, ATF5, Sox-2 and PAX-6, and the majority of cells do not exhibit one or more markers for undifferentiated hBS cells, such as, e.g., SSEA-3, SSEA-4, GCTM-2, Tra-1-60, Tra-1-80, Oct-4, Cripto, Rex-1 or FGF4.

Another embodiment relates to a preparation of neural progenitor cells obtained by a method as described herein, wherein the amount of neural progenitor cells may be at least 50% of the total cell population, such as, e.g. at least 60%, at least 70%, at least 80%, at least 90%, at least 95%, at least 96%, at least 97%, at least 98%, at least 99% or 100%.

Differentiation Method

The neural progenitor cells may also be differentiated as described above. Thus, the invention also relates to the use of differentiated cells derived from the neural progenitor cells obtained by the method described herein for the manufacture of a medicament for the prevention or treatment of pathologies and/or diseases caused by tissue degeneration. such as, e.g., the degeneration of neural tissue.

The invention also relates to the use of the differentiated cells for the manufacture of a medicament for the prevention or treatment of pathologies and/or diseases in the nervous system.

Examples of diseases, which may be prevented and/or treated by a medicament comprising neural progenitor cells or differentiated cells may be selected from the group consisting of multiple schlerosis, spinal chord injury, encephalopathies, Parkinson's disease, Alzheimer's disease, Huntingdon's disease, stroke, traumatic brain injuries, hypoxia induced brain injuries, ischemia induced brain injuries, hypoglycemic brain injuries, degenerative disorders of the nervous system, brain tumors and neuropathies in the peripheral nervous system.

One method of differentiating the progenitor cells comprises dissociating the cells by enzymatic or mechanical treatment and subsequently seeding the progenitor cells on a support substrate that promotes differentiation into mature neurons and/or glial cell types. An example of a suitable support substrate for differentiation of the neural progenitor cells is a polyornithin/laminin or laminin support substrate in combination with the withdrawal of growth factors FGF and EGF and/or the addition of various differentiating factors in various combinations, like SHH (sonic hedgehog), NTN (neurturin), Interleukin-11, Interleukin-1β, TGF-β3 (transforming growth factor-β3), HGF (hepatocyte growth factor), β-NGF (β-nerve growth factor), FGF-8, PDGF-BB (platelet-derived growth factor), TGF-β1 (transforming growth factor-β1), GDNF (glial-derived neurotrophic factor), TGF-α, PDGF-AA, Heparin, Ascorbic acid(AA), Retinoic acid (RA), Cystatin C, LIF, MFUdR/Urd, Cytosine B arabinofuranoside, FGF1, TPA, Dopamine (DA), IBMX, Forskolin, db-cAMP, NT-3, BDNF, CNTF, IGF-1, Noggin, and/or BMPs.

The growth medium used for differentiation can be either hBS cell medium, VitroHES™ medium or neural cell medium. Specific examples of differentiation procedures are shown in Example 5.

The differentiated cells may display the expression of at least one of the following neuronal cell type markers, including neuron-specific β-III tubulin (TUJ1), NeuN, DoubleCortin, tyrosine hydroxylase, Map 2, NF-L, NH-H, NSE, DBH, GABA, synaptophysin, glutamate serotonin, Nurr-1, NF200, or NF68, or glial markers, including RIP, GaIC, GFAP, or S-100.

In one aspect of the invention the progenitor cells are differentiated into oligodendrocytes, which can be characterized by the presence of cell markers such as RIP or GaIC.

In another aspect, the progenitor cells are differentiated into astrocytes, which may be characterized by the presence of cell markers such as GFAP and S-100.

The invention further relates to an essentially pure preparation of differentiated progenitor cells, wherein the cells display the expression of at least one of the following neuronal cell type markers, including neuron-specific β-III tubulin (TUJ1), NeuN, DoubleCortin, tyrosine hydroxylase, Map 2, NF-L, NH-H, NSE, DBH, GABA, synaptophysin, glutamate, serotonin, Nurr-1, NF200, or NF68 and/or markers for mature glial cells like GFAP, S-100, GaIC, or RIP.

Other embodiments of the invention appear from the appended claims. The details and particulars described above and in the claims and relating to the methods according to the invention apply mutatis mutandis to the other aspects of the invention.

The invention is further illustrated by the following figures:

### FIGURE LEGENDS:

FIG. 1: Undifferentiated hBS cells cultured on Matrigel™ in serum free hBS medium conditioned for one day with mouse embryonic fibroblast feeder cells (k-hBS medium), sterile filtered (0.22 μm) and supplemented with 4 ng/mL FGF upon use. Light microscopy images of a colony in 10× (A) and 20× (B) magnification. C; Immunocytochemistry showing positive staining for the primary antibody stage specific embryonic antigen 4 (SSEA-4; secondary antibody-FITC). The SSEA-4 antibody is specific for undifferentiated ES cells (40× magnification). D; Positive staining for alkaline phosphatase (20× magnification).

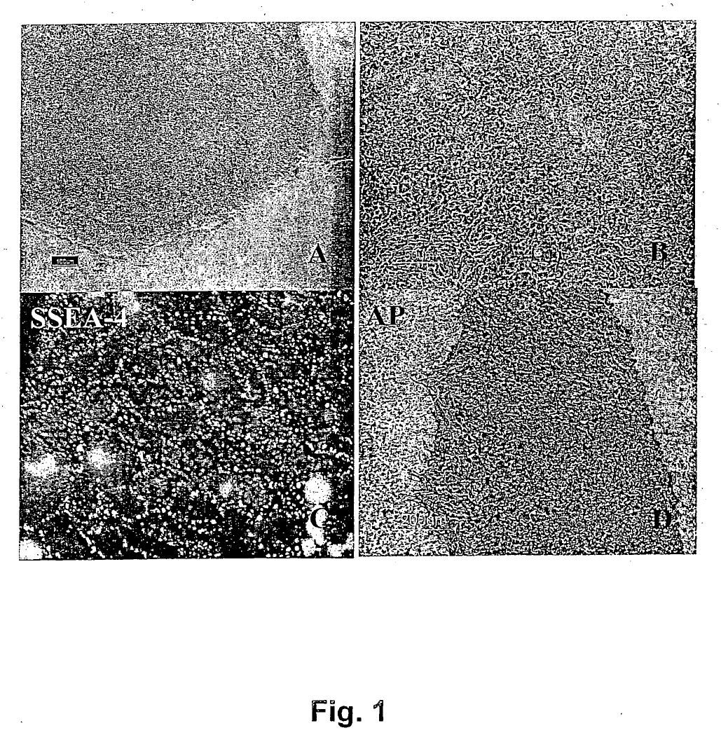

FIG. 2: Neural progenitor induction. Light microscopy image of hBS cells cultured for 4 days on laminin (5 μg/mL; A and B) in k-hBS medium (conditioned with mouse feeder cells). C; hBS cultured for 4 days on gelatin (0.1%) in hBS medium (10× magnification). Immunostaining of cells positive for neuronal progenitor marker intemexin (D) and “neural” progenitor marker nestin (E), (10× magnification).

FIG. 3: Light microscopy image of neural progenitor cells derived from hBS cells, cultured on gelatin (0.1%; A and B) or polyomithine (10 μg/mL)/laminin (5 μg/mL; C) in hBS medium supplemented with FGF (4 ng/mL) for 6 days (10× magnification). Progenitors are growing in neurosphere like formations surrounded by more differentiated cells with a possible non-neural cell fate. D and E showing positive staining of these progenitor rosette formations for the early neuronal marker β_{III}-tubulin (10× magnification).

FIG. 4: Light microscopy images showing neural progenitors growing in a monolayer after the first passage. Progenitors are cultured on gelatin (0.1%) in hBS medium supplemented with 4 ng/mL of FGF. A and B is in 10× magnification, and C and D in 20× magnification.

FIG. 5: Mature neurons and glia cells appeared in large numbers in cultures after just one week of differentiation. For differentiation the progenitors were grown on polyornithine (10 μg/mL)/laminin (5 μg/mL) in neural cell medium in the absence of FGF and EGF for 7 days after passaging. The differentiated progenitor cells were immunoreactive for neural marker MAP2ab (A), astrocyte marker GFAP (B; 40× magnification).

FIG. 6: Differentiated neural progenitors immunoreactive for the midbrain dopaminergic marker tyrosine hydroxylase (A) are shown in 40× magnification. Differentiation was induced by culturing the progenitors on laminin (5 μg/mL) in hBS medium supplemented with TGF-β1 (10 ng/mL) for 7 days. B; MAP2ab positive neural cells generated under the same conditions. C; Negative immunoreactivity of these cells for undifferentiated hBS cell marker SSEA-3 and in D; Hoechst nuclei staining (40× maginification). B, C and D are from the same view.

FIG. 7: A flow chart describing the method for generation of neural progenitor cells from feeder free (Matrigel™) cultures of undifferentiated hBS cells.

FIG. 8: Human hBS cell and neural progenitor (NP) cell morphology at different passages. Light microscopy images of hBS cells cultured under feeder free conditions on Matrigel™ in k-hBS medium (+4 ng/mL FGF; a), NPs in passage 1 (p.1) 2 days after plating (b), 6 days after plating (c), NPs in p.3 (d, e), NPs in p.7 (f), NPs in p.9 (g). The progenitors were derived and propagated according to the method in FIG. 7 and example 4.

FIG. 9: Oct-4 declines with increased passage number and Sox2 is expressed in neural progenitors (NPs). RT-PCR of NPs in different passages (e. g. passage 1 to passage 9) for markers; (A) Oct-4 expression; lane 1: 100 bp DNA ladder; lane 2: Undifferentiated hBS cell on Matrigel™; lane 3: Adult hippocampal progenitors from raqt (AHP; control); lane 4: NP passage 1; lane 5: NP passage 3; lane 6: Negative control; lane 7: 100 bp DNA ladder; lane 8: hBS cells; lane 9: NP passage 1; lane 10: NP passage 3; lane 11: NP passage 7; lane 12: NP passage 9; lane 13: Positive control. (B) Husox2 and GAPDH; lane 1: 100 bp DNA ladder; lane 2: hBS cells; lane 3: AHPs; lane 4: NP passage 1; lane 5: NP passage 3; lane 6: NP passage 7; lane 7: NP passage 9; lane 8 Positive control; lane 9: Negative control. The progenitors were derived and propagated according tp the method in FIG. 7 and example 4. AHP; adult hippocampal progenitors, pC; positive control eg. human genomic DNA, nC; negative control (water instead of sample cDNA).

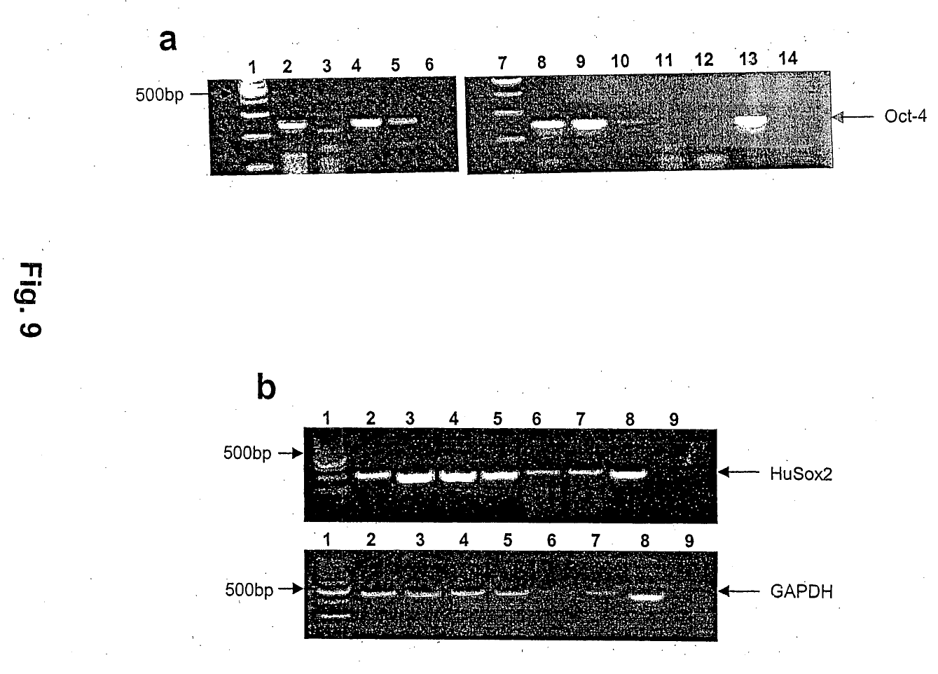

FIG. 10: Mesodermal and endodermal expression declines with increasing passage number in neural progenitor (NP) cultures. RT-PCR of NPs for the expression of markers; Desmin (mesoderm; a) and α-Fetoprotein (AFP; endoderm; b). The progenitors were derived and propagated according to the method in FIG. 7 and example 4. (A) Desmin expression. (B) AFP expression. Common for (A) and (B): lane 1: 100 bp DNA ladder; lane 2: hBS cells; lane 3: AHPs (adult hippocampal progenitors); lane 4: NP passage 1; lane 5: NP passage 3; lane 6: NP passage 7; lane 7: NP passage 9; lane 8: Positive control (human genomic DNA); lane 9: Negative control.

FIG. 11: Neural progenitors (NPs) express immature neural/neuronal/glial markers. NPs immunoreactive for antibodies against Oct-4 in passagel (a), Nestin in passagel (b), A2B5 in passage3 (c), NCAM (green) and Oct-4 in passage2 (red; d), Nestin in passage 3 (e), Nestin (red) and Histone H3 in p.3 (green; f), Nestin (red) and Histone H3 in p.7 (green; g), Internexin in p.7 (h), NCAM in p.9 (i). Blue staining in all figures is the nuclei staining Hoechst. The progenitors were derived and propagated according tp the method in FIG. 7 and example 4.

FIG. 12: Differentiated neural progenitors (NPs), derived and propagated according tp the method in FIG. 7 (example 4), express markers for all three neural lineages. The NPs differentiated into GFAP, (astrocytes; a), MAP2ab (neurons; b) and GaIC (oligodendrocytes; c) expressing cells. Hoechst nuclei staining (blue).

The invention will now be described with reference to the following examples. The examples are included herein for illustrative purposes only and are not intended to limit the scope of the invention in any way. The general methods described herein are well known to a person skilled in the art and all reagents and buffers are readily available, either commercially or easily prepared according to well-established protocols in the hands of a person skilled in the art. All incubations were in 37° C., under a 5% CO₂ atmosphere and 95% humidity.

### EXAMPLES

### Example 1

### Generation and Propagation of Neural Progenitor Cells

Materials and Methods

hBS cells cultured under feeder free conditions were enzymatically dissociated, using collagenase type IV (200 U/mL) for a 15 to 20 minute incubation, to generate a cell suspension of small cell aggregates and single cells. The cell suspension was washed, pelleted, resuspended and plated onto laminin (5 μg/mL in PBS), gelatin (0.1% in H₂O), polyornithine (10 μg/mL in H₂O) or polyornithine/laminin coated Primaria cell culture plastic plates/flasks (Falcon) in BS cell medium; unconditioned or conditioned with mouse feeder cells and supplemented with 4 ng/mL FGF-2, or neural cell medium with or without FGF-2 (20 ng/mL) and EGF (20 ng/mL; Gibco, Gaithersburg, Md.). Neural cell medium consists of Dulbecco's minimal essential medium (DMEM)/F12 (1:1), N2-supplement (1:100), L-glutamine (2 mM), and penicillin/streptomycin (100 units/mL; Gibco, Gaithersburg, Md.). Fifty percentage of the medium was changed every second to third day. The cells were cultured under these conditions for 6 to 8 days then pasaged using collagenase type IV and re-seeded under the same conditions. After the first passage progenitors generated were passaged every 3 to 6 days or at 70 to 80% confluence.

Freezing and Thawing:

The neural progenitor cells were dissociated by incubating with collagenase type IV (200 U/mL) solution for 15 minutes. The cell suspension was then collected, diluted in culturing medium (37° C.), pelleted, washed in culturing medium (37° C.) and resuspended in freeze-medium (4 to 8° C.). The freeze-medium consisted of culturing medium supplemented with 10% DMSO. The cells were frozen at a cell density of 1 million cells/mL. The cell suspension was aliquoted in 1.8 mL Nunc CryoTubes (Nalge Nunc International, Rochester, N.Y.) and frozen slowly (−1° C./min) at −80° C. overnight or at least for 2 h, then transferred to a liquid nitrogen tank for prolonged storage. Thawing of the cells was done by a rapid thawing by placing the CryoTubes in 37° C. water bath until completely thawed, transferring the suspension to preheated (37° C.) culturing medium for 5 min, pellet cells, wash in culturing medium (37° C.) and resuspend in culturing medium. The thawed cells were then seeded, as described above for propagation of progenitor cells.

Results

Induction of Neural Cell Fate in hBS Cells and Characterisation;

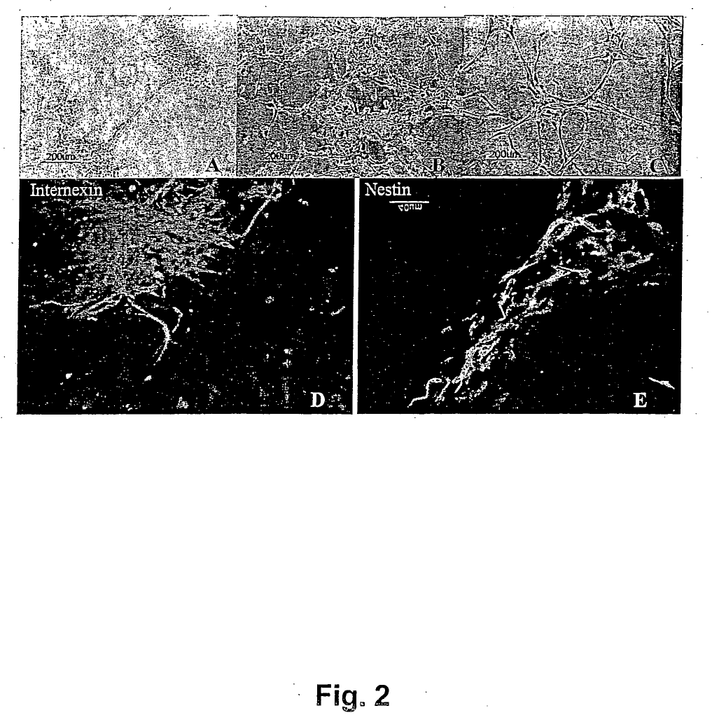

Undifferentiated feeder free cultures of hBS cells were enzymatically dissociated to a cell suspension of small cell aggregates and single cells, and plated onto various coatings (support substrates) in different growth media and supplemented with various growth factors. After 3 to 4 days, neurosphere-like colonies were generated. The cells formed 3 dimentional colonies with elongated cells forming star-shaped patterns resembling neurosphere rosette formations (FIG. 2).

Different coatings and medium components were used to determine the most efficient combination for generation of neural progenitor cells. Polyornithine coating alone was the least adherent and differentiating surface. Gelatine and laminin coating demonstrated the best adhesion and pre-differentiation properties. Even at this early this stage, immunore-activity for the “neuroectodermal” marker nestin (an intermediate-filament protein) and the immature neuronal marker internexin was observed indicating a neural cell fate (FIG. 2).

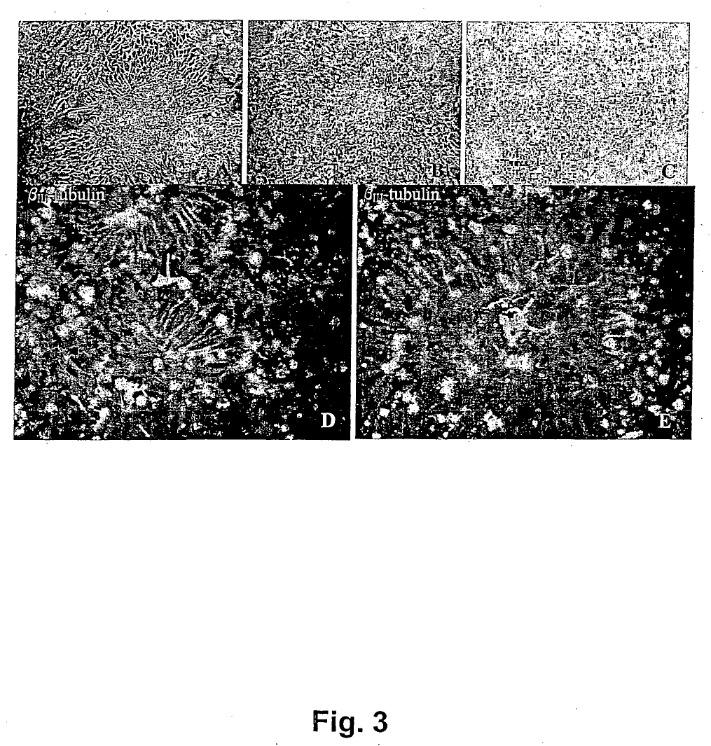

After a further two days of culturing under these conditions (6 days in total), colonies with neurosphere-like appearance (FIG. 3) grew larger. These cells were positive for the progenitor cell marker nestin, and the immature neuronal marker internexin (data not shown). Cells positive for the early neuronal marker, β_{III}-tubulin were also generated (FIG. 3). These “neurospheres” were surrounded by cells with diverse morphologies, probably representing differentiated cells from all three neural lineages and also some contaminating cells, pre-differentiated towards cell types of all three embryonic germ layers. No immunoreactivity for SSEA-3 and -4 or GCTM-2 markers, which identify undifferentiated hBS cells, was observed (data not shown). This, together with positive staining for nestin and internexin, demonstrates that these cells are no longer undifferentiated hBS cells, but progenitors committed to a neural cell fate.

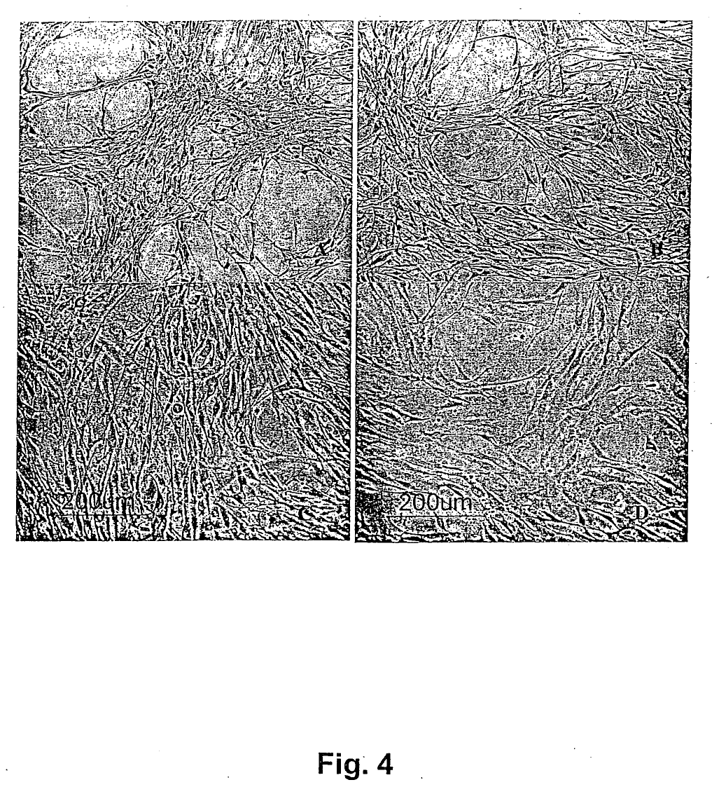

Supplementing the medium with FGF-2 and epidermal growth factor (EGF), a growth factor combination that is known to be effective for the propagation of human fetal- and adult-derived neuroepithelial progenitors, facilitated sequential propagation and expansion of the neurosphere-like cultures. The neural progenitor cultures were allowed to expand for another few days, before immunocytochemistry again was performed using a broad range of neuroectodermal markers. At this time the wells were almost confluent. The same status of immunoreactivity was observed as described just above (nestin, intemexin, β_{III}-tubulin; data not shown). Furthermore, large areas with mature neural cells could be observed showing positive staining for microtubule-associated protein 2 (MAP2ab), glial fibrillary acidic protein (GFAP), and galactocerebroside (GacC), demonstrating a rapid generation of mature cells of the all three neural lineages (8 to 10 days; data not shown). This early differentiation into mature neurons, astrocytes and oligodendrocytes occurred mostly in progenitor cultures on polyornithine- and laminin-coatings. Cultures on gelatine mostly sustained a progenitor cell population. This thus shows that the progenitor population generated is of neural character. After the first passage, the progenitor populations were passaged every 3 to 6 days. Progenitor populations cultured on gelatin (0.1%) in hBS medium (4 ng/mL FGF-2) were maintained for up to 14 passages (6 to 12 weeks). These neural progenitors could be frozen and later thawed again, while maintaining their proliferating ability and potential to differentiate further to mature neurons and glia. A survival rate of around 80% was obtained after thawing the cells. The progenitor populations were, after the first passage, growing as more of a monolayer with networks of cells with neural-like morphology (FIG. 4).

Proof of Proliferation

The progenitor cells were proliferating with a doubling time of around 2 days, suggesting that the population consisted of progenitor cells, as opposed to terminally differentiated cells.

### Example 2

### In vitro Differentiation of Neural Progenitors as Generated in Example 1

Material and Methods

Progenitors were passaged by collagenase IV incubation and seeded on polyornithine/laminin or laminin coated Primaria cell culture plastic plates in neural cell medium or hBS medium without the addition of any growth factors. In some of the differentiation experiments ascorbic acid (100 or 200 μg/mL), or growth factor PDGF (2 ng/mL), TGF-β1 (10 ng/mL), GDNF (2 ng/mL), with or without EGF (20 ng/mL; Gibco, Gaithersburg, Md.) was added to the medium. The progenitors were left to differentiate for 7 to 10 days before fixation and immunocytochemistry was performed.

Results

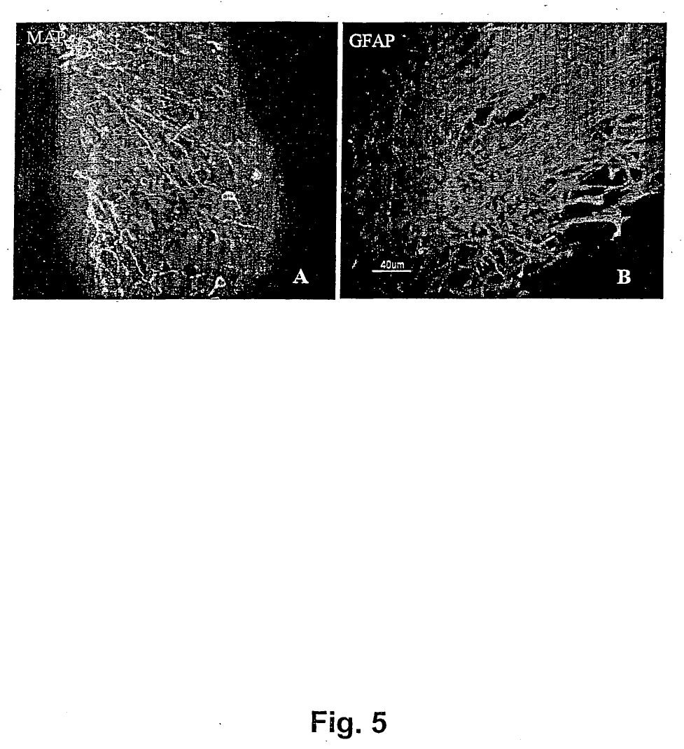

Progenitors differentiated on polyomithine/laminin in neural cell medium without growth factors showed positive staining for a neuronal cell marker (MAP2ab) and an astrocyte marker (GFAP) were observed in very large numbers (FIG. 5) after only 7 days of differentiation. Some cells positive for the oligodendrocyte marker GaIC were also detected (data not shown). The cultures displayed the morphology characteristic of mature neurons and glia. These results show that the expanded populations of neural progenitors have the capacity to generate derivates of all three fundamental neural lineages in vitro.

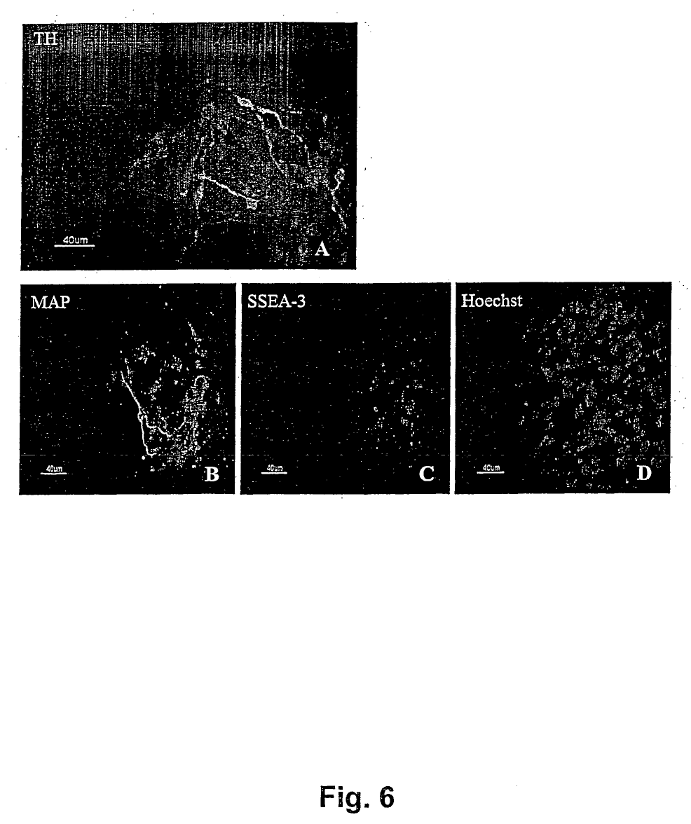

Other differentiation experiments were performed for 7 days on laminin in hBS cell medium supplemented with TGF-β1. These cells expressed immunoreactivity to the dopaminergic marker tyrosine hydroxylase (FIG. 6A), as well as MAP2ab (FIG. 6B) and GFAP. No detection of cells positive for undifferentiated hBS cell markers or neural progenitor cell markers were observed after differentiation (FIG. 6C).

### Example 3

### Immunocytochemistry Analysis, Proliferation Assay for Neural Progenitor Cells

Immunocytochemistry Analysis

Immunostainings of undifferentiated hBS cells to evaluate the expression of SSEA-1, -3, and -4 was performed as described (Thomson et al, 1998), and AP staining according to Xu et al, 2001. Standard protocols were used for immunophenotyping of neural progenitor to cells and for differentiated cells. Fixation with 4% paraformaldehyde was used unless otherwise specified. Primary antibodies included; mouse monoclonal anti-Nestin (1:500; Pharmingen), α-Intemexin (1:750; Chemicon), βIIII-tubulin (1:100; Sigma, St Louis, Mo., USA), rat anti-stage specific embryonic antigen-3 (SSEA-3; 1 μg/mL; DSHB, University of Iowa, USA), and Mouse anti-SSEA4 (1 μg/mL; DSHB), Mouse Monoclonal anti-MAP2ab (1:100; Sigma), rabbit anti-cow GFAP (1:500; DAKO, Glostrup, Denmark), Rabbit anti-GaIC (1:75; Sigma), and monoclonal mouse anti-TH (1:2000; Sigma), monoclonal mouse anti-A2B5 (1:200; Chemicon, Temecula, Calif.), polyclonal rabbit anti-NCAM (1:200; Chemicon), monoclonal mouse anti-α-fetoprotein (AFP, 1:200; Sigma), Tra-1-81 (1:200; Santa Cruz, SDS), polyclonal rabbit anti-human cardiotrophin-1 (1:200; Chemicon) and polyclonal rabbit anti-Phospho-Histone H3 (1:150; KeLab, Upstate). Detection of primary antibody was performed by using goat anti-mouse Alexa fluor 488 IgG (1:4000), goat anti-rabbit Alexa Fluor 594 (1:4000; Molecular Probes), sheep anti-rabbit Fluorescein (FITC; 1:200; Chemicon), donkey anti-mouse FITC (1:200; Termo), donkey anti-rat FITC (1:200; Jackson Laboratories, West Grove, Pa.), and donkey anti-mouse Texas Red (TxR; 1:2000; Jackson). The nuclear dye bisbenzimide was used from a stock solution at 2.5 μg/ml (1:80, Hoechst 33258, Sigma-Aldrich Sweden AB). Primary antibodies were incubated over night at 4° C. or at room temperature for 1 h. Proper controls for primary and secondary antibodies revealed neither non-specific staining nor antibody cross-reactivity. Expression of alkaline phosphatase (AP) in hES cells was detected following fixation of cells with citrate-acetone-formaldehyde fixative solution using Sigma diagnostics kit (Sigma) according to the manufacturer's instruction.

Calculation of Doubling Time

Neural progenitors were counted using a Bürken chamber when passaging. Doubling time was calculated by comparing the number of cells seeded to the number of cells at the following passage.

### Example 4

### The Gelatin Method for Generation of Neural Progenitors

Material and Methods

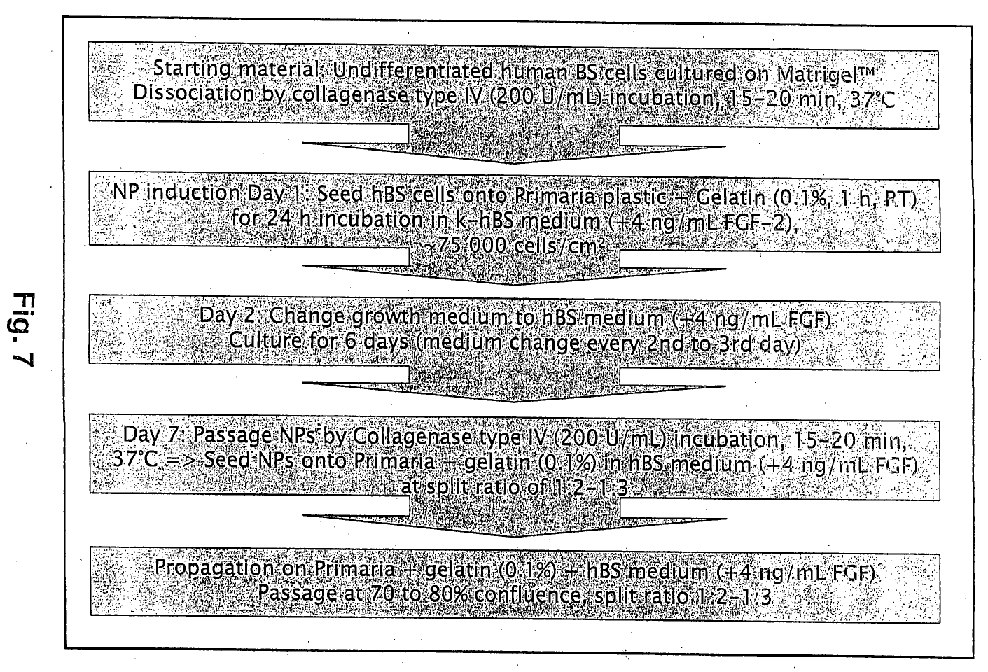

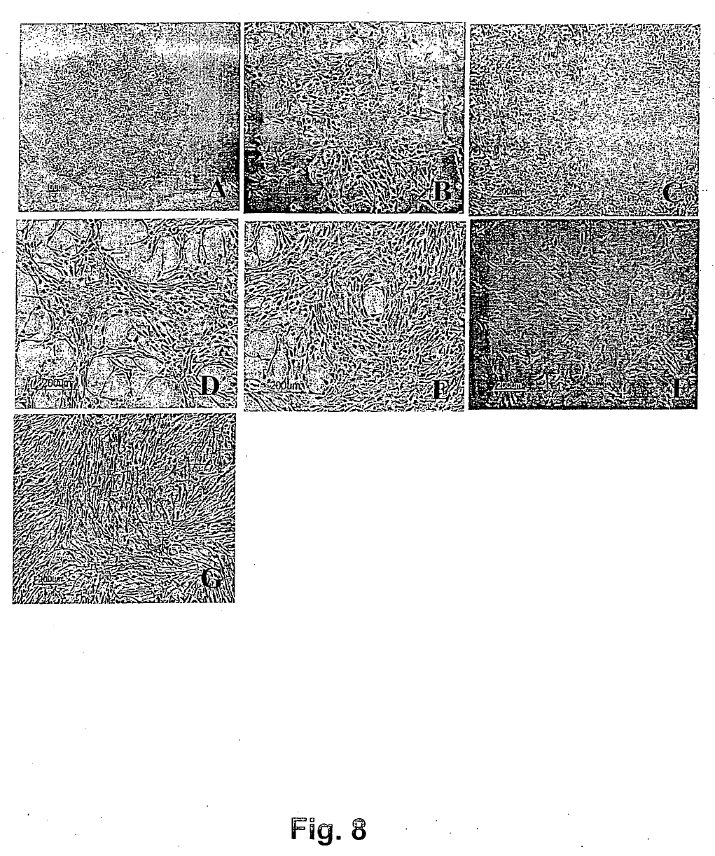

Gelatin (0.01 to 0.1%) and hBS cell medium was found to be the most efficient conditions for the generation and propagation on neural progenitor cells from undifferentiated feeder free (Matrigel™) cultured of hBS cells. The method for the neural progenitor generation and propagation is described in brief in FIG. 7. Undifferentiated feeder free cultures of hBS cells were dissociated by collagenase type IV treatment for 15 to 20 minutes at 37° C. The cell suspension was further dissociated with a pipette to a mixture of single cells and small cell aggregates (of 20 to 100 cells). The cell suspension was then diluted in warm (37° C.) media, pelleted (1500 rpm, 5 minutes), washed, pelleted and resuspended in culturing medium. The cell suspension was seeded onto gelatin (0.1%) coated Primaria plastic culture plates (Falcon) in k-hBS medium supplemented with 4 ng/mL of FGF-2. The following day, the culturing media was changed to unconditioned hBS medium. The cells were left to differentiate under these conditions for 6 days (FIG. 28). Half of the culturing media was changed every second to third day. The cells were passaged on day 7 using collagenase type IV treatment for a 15 to 20 minute incubation. The cells were split at a ratio of 1:2 to 1:3 and seeded onto gelatin (0.1%) coated Primaria plates in unconditioned hBE medium (including 4 ng/mL FGF-2). After this first passage on gelatin the progenitors grew in a monolayer (FIG. 8) and were passaged every 3 to 6 days or when cells reached 70 to 80% confluence.

Results

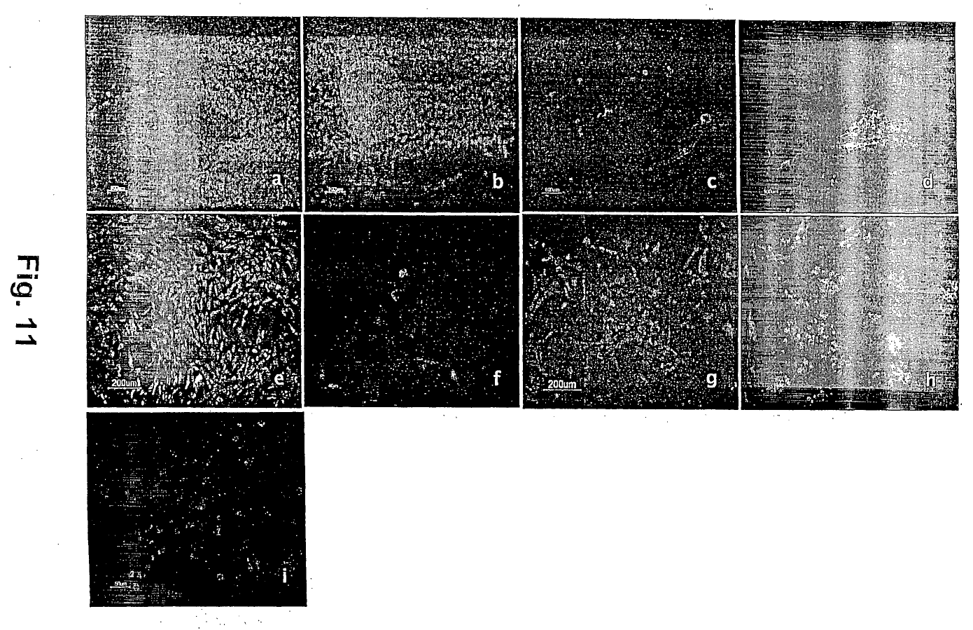

FIG. 28 shows the neural progenitor cell morphology at different passages. The progenitors were cultured on gelatin (0.1%) in hBS medium supplemented with 4 ng/mL of FGF-2 (according to FIG. 27). The progenitors generated by this method express the hBE cell marker Oct-4 in early passages. The expression declines after passage 3. The progenitors express Sox2 (a neural progenitor marker) in passage 1 to 9 at least. The progenitor populations express Desmin (marker for mesoderm) in passage 1 and 3 and the endoderma marker AFP (alfa-fetoprotein) only in passage 1. In FIG. 29 and FIG. 30 RT-PCR analysis from neural progenitors in different passages, established and propagated according to FIG. 7, are shown. The immunocytochemistry analysis of these progenitor populations revealed the expression of Oct-4 (marker for undifferentiated hBS cells) only in early passages. The progenitors express nestin (marker for neural progenitor cells), NCAM (marker for neuronal progenitors and neurons), Internexin (marker for neuronal progenitor cells), A2B5 (glial progenitors and type II astrocytes) as well as the mitotic marker Histone H3. In FIG. 11 the immunocytochemistry results for neural progenitors in different passages, is demonstrated.

### Example 5

### Differentiation of Neural Progenitors Derived and Propagated According to the Method in Example 4

Material and Methods

Neural progenitors were derived and propagated according to example 4 (“the gelatin method for generation of neural progenitors”). For differentiation, the progenitors were passaged by enzymatic treatment using collagenase type IV for 15-20 minutes at 37° C. The cell suspension was seeded onto gelatin and/or laminin coated Primaria plastic surfaces in hBS medium without FGF addition. Differentiation was induced over 8 to 14 days and then analyzed by antibody staining (immunocytochemistry).

Results

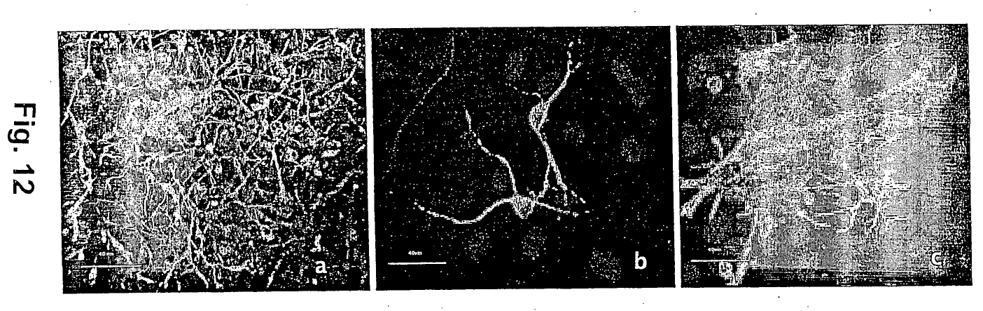

These differentiated neural progenitors expressed markers for all three neural lineages (FIG. 12) according to immunocytochemical analysis. Differentiated cells, positive for the neuronal marker MAP2ab, the astrocyte marker GFAP and the oligodendrocyte marker GaIC, were detected (FIG. 12).

### Example 6

### RT-PCR Analysis of Neural Progenitor Cells (Method)

Total RNA extraction; Qiagen RNeasy® Mini Kit according to manufacturer's instructions. cDNA synthesis; M-MLV reverse transcriptase (Promega, Madison, Wiss.) and RT-PCR by TaqBead™ Hot Start Polymerase according to manufacturer's instructions. RT-PCR primers (Cybergene): Oct-4 (247 bp); sense CGTGAAGCTGGAGAAGGAGAAGCTG, antisense CMGGGCCGCAGCTTACACATGTTC (55° C., 30 cycles); HuSox2 (318 bp); sense CGGAAAACCMGACGCTCA; antisense GCCGTTCATGTAGGTCTGCG (55° C., 35 cycles); Desmin (402 bp); sense CAGGGACATCCGGGCTCAGTAT; antisense AGCTTCCGGTAGGTGGCAATCT (step down 58° C.-50° C., 30 cycles); AFP (453 bp); sense ACTCCAGCATCGATCCCACTTT; antisense AGCTTCCGGTAGGTGGCAATCT (step down 58° C.-50° C., 30 cycles); GAPDH (451 bp); sense ACCACAGTCCATGCCATCAC; antisense TCCACCACCCTGTTGCTGTA (54° C., 30 cycles).

### Method for Establishing hBS Cells Suitable for Use in a Method of the Present Invention

In PCT application published as WO 03/055992 (to the same Applicant) on 10 Jul. 2003, i.e. after the priority date of the present invention, a suitable method for establishing hBS cells is described. In one aspect of the present invention, the cells employed are obtained by the method claimed in WO 03/055992, which is hereby incorporated by reference.

The method for establishing pluripotent human blastocyst-derived stem cells or cell line from a fertilized oocyte comprises the steps of
i) using a fertilized oocyte optionally, having a grade 1 or 2, to obtain a blastocyst, optionally having a grade A or B,
ii) co-culturing the blastocyst with feeder cells for establishing one or more colonies of inner cell mass cells,
iii) isolating the inner cell mass cells by mechanical dissection,
iv) co-culturing of the inner cell mass cells with feeder cells to obtain a blastocyst-derived stem cell line.
v) optionally, propagation of the blastocyst-derived stem cell line.

As a starting material for this procedure, fertilized oocytes are used. The quality of the fertilized oocytes is of importance for the quality of the resulting blastocysts. The human blastocysts in step i) of the method may be derived from frozen or fresh human in vitro fertilized oocytes. In the following is described a procedure for selecting suitable oocytes for use in a method according WO 03/055992. It was found that an important success criterion for the present method is a proper selection of oocytes. Thus, if only grade 3 oocytes are applied, the probability of obtaining a hBS cell line fulfilling the general requirements (described below) is low.

Donated fresh fertilized oocytes: On day 10 the oocyte is aspirated in Asp-100 (Vitrolife), and fertilized on day 1 in IVF-50 (Vitrolife). The fertilized oocyte is evaluated based on morphology and cell division on day 3. The following scale is used for fertilized oocyte evaluation:
Grade 1 fertilized oocyte: Even blastomers, no fragments
Grade 2 fertilized oocyte: <20% fragments
Grade 3 fertilized oocyte: >20% fragments

After evaluation on day 3, fertilized oocytes of grade 1 and 2 are either implanted or frozen for storage. Fertilized oocytes of grade 3 are transferred to ICM-2 (Vitrolife). The fertilized oocytes are further cultured for 3-5 days (i.e. day 5-7 after fertilization). The blastocysts are evaluated according to the following scale:
Grade A Blastocyst: Expanded with distinct inner cell mass (ICM) on day 6
Grade B Blastocyst: Not expanded but otherwise like grade A
Grade C Blastocyst: No visible ICM

Donated frozen fertilized oocytes: At day 2 (after fertilization) the fertilized oocytes are frozen at the 4-cell stadium using Freeze-Kit (Vitrolife). Frozen fertilized oocytes are stored in liquid nitrogen. Informed consent is obtained from the donors before the 5-year limit has passed. The fertilized oocytes are thawed using Thaw-Kit (Vitrolife), and the procedure described above is followed from day 2.

As described above, fresh fertilized oocytes are from grade 3 quality, and frozen fertilized oocytes are from grade 1 and 2. According to data obtained by the establisment methods, the percentage of fresh fertilized oocytes that develop into blastocysts is 19%, while 50% of the frozed fertilized oocytes develop into blastocysts. This means that the frozen fertilized oocytes are much better for obtaining blastocysts, probably due to the higher quality of the fertilized oocytes. 11% of the blastocysts derived from fresh fertilized oocytes develop into a stem cell line, while 15% of the blastocysts derived from frozen fertilized oocytes develop into a stem cell line. In summary, of the fertilized oocytes that were put into culture 2% of fresh fertilized oocytes developed into a stem cell line, and 7% of frozen fertilized oocytes that were put into culture developed into a stem cell line.

The culturing of the fertilized oocyte to the blastocyst-stage is performed after procedures well-known in the art. Procedures for preparing blastocysts may be found in Gardner et al, Embryo culture systems, In Trounson, A. O., and Gardner, D. K. (eds), Handbook of in vitro fertilization, second edition. CRC Press, Boca Raton, pp. 205-264; Gardner et al, Fertil Steril, 74, Suppl 3, O-086; Gardner et al, Hum Reprod, 13, 3434,3440; Gardner et al, J Reprod Immunol, In press; and Hooper et al, Biol Reprod, 62, Suppl 1, 249.

After establishment of blastocysts in step i) optionally derived from fertilized oocytes having grade 1 or 2, the blastocysts having grade A or B are co-cultured with feeder cells for establishing one or more colonies of inner cell mass cells. After being plated onto feeder cells, their growth is monitored and when the colony is large enough for manual passaging (approximately 1-2 weeks after plating), the cells may be dissected from other cell types and expanded by growth on new feeder cells. The isolation of the inner cell mass cells is performed by mechanical dissection, which may be performed by using glass capillaries as a cutting tool. The detection of the inner cell mass cells is easily performed visually by microscopy and, according, it is not necessary to use any treatment of the oocytes with enzymes and/or antibodies to impair or remove the trophectoderm.

Thus, the procedure of WO 03/055992 alleviates the need for immunosurgery. By comparing the success-rate in using immunosurgery versus the present method, which leaves the trophectoderm intact, it has been observed that the much simpler, faster and nontraumatic procedure of avoiding immunosurgery is more efficient than immunosurgery. These procedures make the preparation of stem cell lines, and the differentiation of these cell lines commercially feasible. From a total of 122 blastocysts, 19 cell lines were established (15.5%). 42 blastocysts were processed by immunosurgery and 6 of these resulted in successfully established cell lines (14%). Eighty blastocysts were processed by the present method and 13 cell lines were established (16%).

Subsequent to dissection of the inner cell mass, the inner cell mass cells are co-cultured with feeder cells to obtain a blastocyst-derived stem (BS) cell line. After obtaining the hBS cell line, the cell line is optionally propagated to expand the amount of cells. Thus, the blastocyst-derived stem cell line may be propagated e.g. by passage of the stem cell line every 4-5 days. If the stem cell line is cultured longer than 4-5 days before passage, there is an increased probability that the cells undesirably will differentiate.

A specific procedure of passaging the cells in a feeder culture system is given in Establishment example 5 herein.

Human BS cell lines may be isolated either from spontaneously hatched blastocysts or from expanded blastocysts with an intact zona pellucida. In the method described above the blastocyst in step i) is a spontaneously hatched blastocyst. For hatched blastocysts the trophectoderm may be left intact. Either hatched blastocysts or blastocysts with a removed or partially removed zona pellucida may be put on inactivated feeder cells.

The zona pellucida of the blastocyst may be at least partially digested or chemically frilled prior to step ii) e.g. by treatment with one or more acidic agents such as, e.g., ZD™-10 (Vitrolife, Kungsbacka, Sweden), one or more enzymes or mixture of enzymes such as pronase.

A brief pronase (Sigma) treatment of blastocysts with an intact zona pellucida results in the removal of the zona. Other types of proteases with the same or similar protease activity as pronase may also be used. The blastocysts can be plated onto said inactivated feeder cells following the pronase treatment.

In an embodiment of the invention step ii) and/or step iv) may be performed in an agent that improves the attachment of the blastocysts and/or if relevant the inner cell mass cells to the feeder cells. A suitable substance for this purpose is a hyaluronic acid.

A suitable medium for plating the blastocysts onto feeder cells can be hBS-medium that may be complemented with hyaluronic acid, which seems to promote the attachment of the blastocysts on the feeder cells and growth of the inner cell mass. Hyaluronan (HA) is an important glycosaminoglycan constituent of the extracellular matrix in joints. It appears to exert its biological effects through binding interactions with at least two cell surface receptors: CD44 and receptor for HA-mediated motility (RHAMM), and to proteins in the extracellular matrix. The positive effects of HA during the establishment of hBS cells may be exerted through its interactions with the surfactant polar heads of phospholipids in the cell membrane, to thereby stabilize the surfactant layer and thus lower the surface tension of the inner cell mass or blastocyst which may result in increased efficiency in binding to the feeder cells. Alternatively, HA may bind to its receptors on the inner cell mass or blastocyst and/or to the feeder cells and exert biological effects which positively influence the attachment and growth of the inner cell mass. According to this, other agents that may alter the surface tension of fluids, or in other ways influence the interaction between the blastocyst and feeder cells can also be used in instead of hyaluronic acid.

In the method describe above culturing of the feeder cells is of importance for the establishment of the hBS cell line. The propagation of blastocyst-derived stem cell line may comprise passage of the feeder cells at the most 3 times, such as e.g. at the most 2 times.

Suitable feeder cells for use in a method of the invention are fibroblasts of e. g. embryonic or adult origin. In a method according to the invention the feeder cells employed in steps ii) and iv) are the same or different and originate from animal source such as e.g. any mammal including human, mouse, rat, monkey, hamster, frog, rabbit etc. Feeder cells from human or mouse species are preferred.

Another important criterion for obtaining an hBS cell line fulfilling the general requirements are the conditions under which the blastocysts are cultured. The blastocyst-derived stem cell line may accordingly by propagated by culturing the stem cells with feeder cells of a density of less than about 60,000 cells per cm², such as e.g. less than about 55,000 cells per cm², or less than about 50,000 cells per cm². In a specific embodiment, the propagation of blastocyst-derived stem cell line comprises culturing the stem cells with feeder cells of a density of about 45,000 cells per cm². These values apply in those cases where mouse feeder cells are used and it is contemplated that a suitable density can be found for other types of feeder cells as well. Based on the findings of the present inventors, a person skilled in the art will be able to find such suitable densities. The feeder cells may be mitotically inactivated in order to avoid unwanted growth of the feeder cells.

The blastocyst-derived stem cell line obtained by the establishment method described above maintains self-renewal and pluripotency for a suitable period of time and, accordingly it is stable for a suitable period of time. In the present context the term “stable” is intended to denote proliferation capacity in an undifferentiated state for more than 21 months when grown on mitotically inactivated embryonic feeder cells.

The stem cell line obtained by the establishment method described above fulfils the general requirements. Thus, the cell line
i) exhibits proliferation capacity in an undifferentiated state for more than 21 months when grown on mitotically inactivated embryonic feeder cells, and
ii) exhibits normal euploid chromosomal karyotype, and
iii) maintains potential to develop into derivatives of all types of germ layers both in vitro and in vivo, and
iv) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and
v) does not exhibit molecular marker SSEA-1 or other differentiation markers, and
vi) retains its pluripotency and forms teratomas in vivo when injected into immuno-compromised mice, and
vii) is capable of differentiating.

The undifferentiated hBS cells obtained by the method described above are defined by the following criteria; they were isolated from human pre-implantation fertilized oocytes, i.e. blastocysts, and exhibit a proliferation capacity in an undifferentiated state when grown on mitotically inactivated feeder cells; they exhibit a normal chromosomal karyotype; they express typical markers for undifferentiated hBS cells, e.g. OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and do not show any expression of the carbohydrate epitope SSEA-1 or other differentiation markers. Furthermore, pluripotency tests in vitro and in vivo (teratomas) demonstrate differentiation into derivatives of all germ layers.

According to the above, the method provides an essentially pure preparation of pluripotent human BS cells, which i) exhibits proliferation capacity in an undifferentiated state for more than 21 months when grown on mitotically inactivated embryonic feeder cells; ii) exhibits normal euploid chromosomal karyotype; iii) maintains potential to develop into derivatives of all types of germ layers both in vitro and in vivo; iv) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2 v) does not exhibit molecular marker SSEA-1 or other differentiation markers, and vi) retains its pluripotency and forms teratomas in vivo when injected into immuno-compromised mice, and vii) is capable of differentiating.

Procedures for the detection of cell markers can be found in Gage, F. H., Science, 287:1433-1438 (2000). These procedures are well known for the skilled person and include methods such as RT-PCR or immunological assays where antibodies directed against the cell markers are used. In the following, methods for detection of cell markers, hybridisation methods, karyotyping, methods for measuring telomerase activity and teratoma formation are described. These methods can be used to investigate whether the hBS cells obtained according to the establishment method fulfil the above-mentioned criteria.

Immunohistochemistry

The hBS stem cells maintained in culture are routinely monitored regarding their state of differentiation. Cell surface markers used for monitoring the undifferentiated hBS cells are SSEA-1, SSEA-3, SSEA-4, TRA-1-60, TRA-1-81. Human BS stem cells are fixed in 4% PFA and subsequently permeabilized using 0.5% Triton X-100. After washing and blocking with 10% dry milk the cells are incubated with the primary antibody. After extensive washes the cell are incubated with the secondary antibody and the nuclei are visualized by DAPI staining.

Alkaline Phosphatase

The activity of alkaline phosphatase is determined using a commercial available kit following the instructions from the manufacturer (Sigma Diagnostics).

Oct-4 RT-PCR

The mRNA levels for the transcription factor Oct-4 is measured using RT-PCR and gene specific primer sets (5′-CGTGAAGCTGGAGMGGAGAAGCTG, 5′-CAAGGGCCGCAGCTTACACATGTTC) and GAPDH as housekeeping gene (5′-ACCACAGTCCATGCCATCAC, 5′-TCCACCACCCTGTTGCTGTA).

Fluorescence In Situ Hybridization (FISH)

In one round of FISH one ore more chromosomes are being selected with chromosome specific probes. This technique allows numerical genetic aberrations to be detected, if present. For this analysis CTS uses a commercially available kit containing probes for chromosome 13, 18, 21 and the sex chromosomes (X and Y) (Vysis. Inc, Downers Grove, Ill., USA). For each cell line at least 200 nuclei are being analyzed. The cells are resuspended in Camoy's fixative and dropped on positively charged glass slides. Probe LSI 13/21 is mix with LSI hybridization buffer and added to the slide and covered with a cover slip. Probe CEP X/Y/18 is mixed with CEP hybridization buffer and added in the same way to another slide. Denaturing is performed at 70° C. for 5 min followed by hybridization at 37° C. in a moist chamber for 14-20 h. Following a three step washing procedure the nuclei are stained with DAPI II and the slides analyzed in an invert microscope equipped with appropriate filters and software (CytoVision, Applied Imaging).

Karyotyping

Karyotyping allows all chromosomes to be studied in a direct way and is very informative, both numerical and larger structural aberrations can be detected. In order to detect mosaicism, at least 30 karyotypes are needed. However, this technique is both very time consuming and technically intricate. To improve the conditions for the assay the mitotic index can be raised by colcemid, a synthetic analog to colchicin and a microtubule-destabilizing agent causing the cell to arrest in metaphase, but still a large supply of cells are needed (6×10⁶ cells/analysis). The cells are incubated in the presence of 0.1 μg/ml colcemid for 1-2 h, and then washed with PBS and trypsinized. The cells are collected by centrifugation at 1500 rpm for 10 min. The cells are fixed using ethanol and glacial acetic acid and the chromosomes are visualized by using a modified Wrights staining.

Comparative Genomic Hybridization

Comparative genomic hybridization (CGH) is complementary to karyotyping. CGH gives a higher resolution of the chromosomes and is technically less challenging. Isolated DNA is nicktranslated in a mixture of DNA, A4, Texas red-dUTP/FITC 12-dUTP, and DNA polymerase 1. An agarose gel electrophoresis is performed to control the size of resulting DNA fragments (600-2000 bp). Test and reference DNA is precipitated and resuspended in hybridization mixture containing formamide, dextrane sulfate and SSC. Hybridization is performed on denatured glass slides with metaphases for 3 days at 37° C. in a moist chamber. After extensive washing one drop of antifade mounting mixture (vectashield, 0.1 μg/ml DAPI II) is added and the slides covered with cover slips. Slides are subsequently evaluated under a microscope and using an image analysis system.

Telomerase Activity

Since a high activity has been defined as a criterion for hBS cells 6 the telomerase activity is measured in the hBS cell lines. It is known that telomerase activity successively decrease when the cell reaches a more differentiated state. Quantifying the activity must therefore be related to earlier passages and control samples, and can be used as a tool for detecting differentiation. The method, Telomerase PCR ELISA kit (Roche) uses the internal activity of telomerase, amplifying the product by polymerase chain reaction (PCR) and detecting it with an enzyme linked immunosorbent assay (ELISA). The assay is performed according to the manufacturer's instructions. The results from this assay show typically a high telomerase activity (>1) for hBS cells.

The cell lines retain their pluripotency and forms teratomas in vivo when injected into immuno-compromised mice. In addition, in vitro these cells can form hBS cell derived bodies. In both of these models, cells characteristic for all germ layers can be found.

Teratoma Formation in Immunodeficient Mice

One method to analyze if a human BS cell line has remained pluripotent is to xenograft the cells to immunodeficient mice in order to obtain tumors, teratomas. Various types of tissues found in the tumor should represent all three germlayers. Reports have showed various tissues in tumors derived from xenografted immunodeficient mice, such as striated muscle, cartilage and bone (mesoderm) gut (endoderm), and neural rosettes (ectoderm). Also, large portions of the tumors consist of disorganized tissue.

Severe combined immunodeficient (SCID)-mice, a strain that lack B- and T-lymphocytes are used for analysis of teratoma formation. Human BS cells are surgically placed in either testis or under the kidney capsule. In testis or kidney, hBS cells are transplanted in the range of 10 000-100 000 cells. Ideally, 5-6 mice are used for each cell line at a time. Preliminary results show that female mice are more post-operative stable than male mice and that xenografting into kidney is as effective in generating tumors as in testis. Thus, a female SCID-mouse teratoma model is preferable. Tumors are usually palpable after approximate 1 month. The mice are sacrificed after 1-4 months and tumors are dissected and fixed for either paraffin-or freeze-sectioning. The tumor tissue is subsequently analyzed by immunohistochemical methods. Specific markers for all three germlayers are used. The markers currently used are: human E-Cadherin for distinction between mouse tissue and human tumour tissue, α-smooth muscle actin (mesoderm), α-Fetoprotein (endoderm), and β-III-Tubulin (ectoderm). Additionally, hematoxylin-eosin staining is performed for general morphology.

The establishment method is described below in the following “establishment examples”. These examples are included herein for illustrative purposes only and are not intended to limit the scope of the invention in any way. The general methods described herein are well known to a person skilled in the art and all reagents and buffers are readily available, either commercially or easily prepared according to well-established protocols in the hands of a person skilled in the art. All incubations were in 37° C., under a CO₂ atmosphere.

One suitable medium used is termed “BS-cell medium” or “BS-medium” and may be comprised of; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor).

Another suitable medium is “BS cell body medium”, this may be comprised as follows; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine and 100 μM β-mercaptoethanol.

In the present context the term “stable” is intended to denote proliferation capacity in an undifferentiated state for more than 21 months when grown on mitotically inactivated embryonic feeder cells.

### ESTABLISHMENT EXAMPLES

### Establishment Example 1

### Establishment of an Essentially Pure Preparation of Undifferentiated Stem Cells from Spontaneously Hatched Blastocysts

Human blastocysts were derived from frozen or fresh human in vitro fertilized embryos. Spontaneously hatched blastocysts were put directly on feeder cells (EF) in hBS cell medium (KNOCKOUT Dulbecco's Modified Eagle's Medium, supplemented with 20% KNOCKOUT Serum replacement, and the following constituents at the final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor), supplemented with 0.125 mg/ml hyaluronic acid. After plating the blastocysts on the EF cells, growth was monitored and when the colony was large enough for manual passaging approximately 1-2 weeks after plating) the inner cell mass cells were dissected from other cell types and expanded by growth on new EF cells.

### Establishment Example 2

### Establishment of an Essentially Pure Preparation of Undifferentiated Stem Cells from Blastocysts with an Intact Zona Pellucida

For blastocysts with an intact zona pellucida, a brief pronase (10 U/ml, Sigma) incubation in rS2 (ICM-2) medium (Vitrolife, Gothenburg, Sweden) was used to digest the zona, after which the blastocyst was put directly on the EF cell layer in hBS medium supplemented with hyaluronic acid (0.125 mg/ml).

### Establishment Example 3

### Histo-chemical Staining for Alkaline Phosphatase

The cells were harvested for RT-PCR and histological (alkaline phosphatase) and immunocytochemical analysis (see below). RNA isolation and RT-PCR. Total cellular RNA was prepared using Rneasy Mini Kit (Qiagen) according to the manufacturer's recommendations. The cDNA synthesis was carried out using AMV First Strand cDNA Synthesis Kit for RT-PCR (Roche) and PCR using Platinum Taq DNA Polymerase (Invitrogen). Histo-chemical staining for alkaline phosphatase was carried out using commercially available kit (Sigma) following the manufacturer's recommendations.

### Establishment Example 4

### Preparation and Culturing of hBS Cell Line

Mouse embryonic fibroblasts feeder cells were cultivated on tissue culture dishes in EMFI-medium: DMEM (Dulbecco's Modified Eagle's Medium), supplemented with 10% FCS (Fetal Calf Serum), 0.1 μM β-mercaptoehanol, 50 units/ml penicillin, 50 μg/ml streptomycin and 2 mM L-glutamine (GibcoBRL). The feeder cells were mitotically inactivated with Mitomycin C (10 μg/ml, 3 hrs). Human BS cell-colonies were expanded by manual dissection onto inactivated mouse embryonic fibroblasts feeder cells.

Human BS cells were cultured on mitotically inactivated mouse embryonic fibroblasts feeder cells in tissue culture dishes with hBS-cell medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, 4 ng/ml human recombinant bFGF (basic fibroblast growth factor). Seven days after passage the colonies were large enough to generate BS cell bodies.

BS cell colonies were cut with glass capillaries into 0.4×0.4 mm pieces and plated on nonadherent bacterial culture dishes containing hBS cell body medium: KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine and 100 μM p-mercaptoethanol. The BS cell bodies, including cystic BS cell bodies, formed over a 7-9-day period.

### Establishment Example 5

### Passage of hBS Cells

Before passage the hBS cells are photographed using a Nikon Eclipse TE2000-U inverted microscope (10× objective) and a DXM 1200 digital camera. Colonies are passaged every 4-5 days. The colonies are big enough to be passaged when they can be cut in pieces (0.1-0.3×0.1-0.3 mm). The first time the cells are passaged, they have grown for 1-2 weeks and can be cut in approximately four pieces.

The colonies are focused, one by one, in a stereo-microscope and cut in a checkered pattern according to the size above. Only the inner homogeneous structure is passaged. Each square of the colony is removed with the knife, aspirated into a capillary and placed on new feeder cells (with the maximum age of 4 days). 10-16 squares are placed evenly in every new IVF-dish. The dishes are left five to ten minutes so the cells can adhere to the new feeder and then placed in an incubator. The hBS medium is changed three times a week. If the colonies are passaged, medium is changed twice that particular week. Normally a “half change” is made, which means that only half the medium is aspirated and replaced with the equal amount of fresh, tempered medium. If necessary the entire volume of medium can be changed.

### Establishment Example 6

### Vitrification of hBS Cells

Colonies with the appropriate undifferentiated morphology from the cell line are cut as for passage. 100-200 ml liquid nitrogen is sterile filtered into a sufficient amount of cryotubes. Two solutions A and B are prepared (A: 800 μl Cryo PBS with 1 M Trehalose, 100 μl etylen glycole and 100 μl DMSO, B: 600 μl Cryo PBS with 1 M Trehalose, 200 μl etylen glycole and 200 μl DMSO) and the colonies are placed in A for 1 minute and in B for 25 seconds. Closed straws are used to store the frozen colonies. After the colonies have been transferred to a straw, it is immediately placed in a cryotube with sterile filtered nitrogen.

### Establishment Example 7

### Seeding of Embryonic Mouse Feeder (EMFI) Cells

The cells are inactivated with EMFi medium containing Mitomycin C by incubation at 37° C. for 3 hours. IVF-dishes are coated with gelatin. The medium is aspirated and the cells washed with PBS. PBS is replaced with trypsin to detach the cells. After incubation, the trypsin activity is stopped with EMFi medium. The cells are then collected by centrifugation, diluted 1:5 in EMFi medium, and counted in a Bürker chamber. The cells are diluted to a final concentration of 170K cells/ml EMFi medium. The gelatin in the IVF-dishes is replaced with 1 ml cell suspension and placed in an incubator. EMFi medium is changed the day after the seeding.

### A Method for Efficient Transfer of hBS Cells From a Feeder-supported to a Feeder-free Culture System, and Long-term Propagation of hBS Cells Under Feeder-free Conditions

The hBS cells employed in the present invention may be cultured in a feeder-free culture system, which method is advantageous compared to the known methods in that the cells transferred are stable for at least up to 10 passages. Studies by Richards et al. showed that the hBS cell lines could not be propagated in an undifferentiated state for more than six passages on cell-free matrixes, including Matrigel™. However, the hBS cells were stable for up to 35 passages on Matrigel™, still expressing the markers for undifferentiated hBS cells, even after a cycle of freeze/thawing and growth rates remained roughly comparable. Furthermore, a significantly higher number of surviving colonies were observed two days after plating, when mechanical dissociation was compared with enzymatic dissociation. A critical step seems to bee the initial step for transfer of the hBS cells to a feeder-free culture system. Accordingly, below is described a method for transfer of hBS cells to a feeder-free culture system, wherein the hBS cells are mechanically cut from the feeder. In the Feeder-free examples herein, only the centre part of each colony was used, whereas in previous work by Xu et al., the whole colonies were detached by enzymatic treatment with the risk of contaminating the cultures with feeder cells. Furthermore, the use of enzymes, at the very delicate step of transferring the feeder cultured hBS cells to a feeder-free surface, may cause inactivation of important surface molecules involved in cell adhesion and growth. The major components in Matrigel™ are extracellular matrix proteins, like collagen type IV and laminin. Activation of the cell surface integrins upon binding to extracellullar matrix proteins is believed to be a crucial step for the regulation of cell adhesion, survival and proliferation. For example, Integrin alpha 1 has a unique role among the collagen receptors in regulating both in vivo and in vitro cell proliferation in collagenous matrices. Laminin-specific receptors, possibly formed by Integrin α6 and β1 which are highly expressed by hBS cells, may also play a major role in the adhesion of hBS cell to the matrix surface. Thus, one possibility is that some of the important surface receptors for attachment or survival might be negatively affected by the rough initial Collagenase IV treatment before the cells have adapted to the new surface.

In the examples herein different techniques for the transfer of hBS cells to a feeder-free environment were investigated, either by mechanical or enzymatical dissociation, in regards to cell adhesion, survival rate and proliferation. Furthermore, the method was developed in order to facilitate long-term propagation and large-scale production of homogenous populations of undifferentiated hBS cells. The use of conventional cryopreservation techniques for freezing/thawing of the hBS cells was also examined.

Transfer of hBS Cells to Feeder Free Propagation

Subsequent to dissection of the inner cell mass, the inner cell mass cells are co-cultured with feeder cells to obtain a blastocyst-derived stem (BS) cell line. After obtaining the hBS cell line, the cell line is optionally propagated to expand the amount of cells.

Before propagation of the hBS cells in a feeder-free system, the hBS cells may be transferred to a feeder-free system.

As mentioned herein before and as it is demonstrated in the Feeder-free examples a critical factor for the success in the propagation of the hBS cells is the method by which the hBS cells is transferred from a feeder culture system to a feeder-free culture system. Accordingly, the hBS cells must be transferred to the feeder-free culture system by mechanical dissection, which may be performed by using glass capillaries as a cutting tool. As shown in the examples herein, mechanical dissociation resulted in a much more efficient attachment of cells to the Matrigel™, a more rapid proliferation compared to the enzyme treated cultures, and the cells were much more stable during passages. Accordingly, the method for transferring the HS cells according to the invention does not require any enzymatic treatment. As seen in the examples herein, the cells cultured and proliferated under feeder-free conditions have a mitotic index that was similar to that of cells grown under feeder conditions.

The propagation of the blastocyst-derived stem cell line comprises culturing the stem cells under feeder cell free growth conditions, as culturing the hBS cells without feeder cells has a number of advantages, such as, e.g. there is no need for the ongoing production of feeder cells, the production of hBS cells may be easier to scale up to commercial production and there is no risk of DNA transfer or other infection risks from the feeder cells.

Thus, the transfer and propagation step under feeder free conditions may comprise the following steps of
a) transferring the blastocyst derived stem cells from feeder to feeder free culture by mechanical treatment.
b) optionally, culturing the blastocyst derived stem cells under feeder cell free growth conditions in a suitable growth medium and/or on a suitable support substrate, and
c) optionally, passaging the blastocyst derived stem cell line every 3-10 days by enzymatic and/or mechanical treatment.

Normally, all steps i)-iii) are included.

Transfer of hBS Cells From a Feeder Culture System to a Feeder-free Culture System

The transfer step has been found to be a critical step as mentioned above. Accordingly, the transfer should be done by means of mechanically dissociation or mechanical dissection of the cells in the feeder culture system. This mechanical treatment may be done by means of any suitable cutting tool such as a tool having a sharpened end and a size that is appropriate for the cutting. The tool may be made of any suitable material such as, e.g., plastic or glass and an example of a suitable tool is a cutting tool that is a sterile sharpened glass capillary, with a 25 degree angle and a 200 or 300 micrometer lumen, designed for cutting, manipulation, and transfer of hBS colonies, or parts of hBS colonies. It is produced by Swemed Lab International AB, Billdal, Sweden.

The hBS cells to be transferred is a colony of hBS cells and pieces is cut from the centre of the colony and suspended in a suitable medium as cell clusters. The cell clusters are dissociated mechanically one or more times e.g. until the cell clusters have a size that is at least 50% such as, e.g., at the most about 40%, at the most about 30%, at the most about 20%, at the most about 10% or at the most about 5% of that of the original colony. The size is e.g. determined as the diameter of the cluster or colony, respectively.

In the feeder-free examples herein is given suitable conditions for the transfer process. These conditions may of course be varied within appropriate limits, which is within the knowledge of a person skilled in the art.

### Feeder-free Example 1

### Preparation of Conditioned VitroHES™-medium (k-VitroHES™-medium) for Feeder Free Cultures

To prepare mEF cells for conditioning of VitroHES™-medium, a confluent monolayer of mEF cells (passage two) was Mitomycin C treated and seeded in a concentration of 59 000 cells/cm² in a gelatin (0.1%; Sigma) coated culture flask in Dulbecco's Modified Eagle Medium (D-MEM) supplemented with 1% Penicillin/Streptomycin (PEST; 100000 U/ml), 10% Fetal Bovine Serum (FBS) and 2 mM GLUTAMAX™-I Supplement (200 mM); all from GibcoBRL/Invitrogen, Carlsbad, Calif., USA. After a 24 hour incubation period and one wash with PBS (GibcoBRl/Invitrogen), the medium was discarded and replaced with VitroHES™-medium (0.28 mU cm²) for a 24 hour conditioning period. The conditioned VitroHES™-medium (k-VitroHES™-medium) was collected every day up to three times from the same mEF culture (in passage two) and sterile filtered by using a 0.2 μm low protein binding filter (Sarstedt, Landskrona, Sweden). The k-VitroHES™-medium was used either fresh or after freezing at −20° C. and supplemented with 4 ng/ml of bFGF (GibcoRL/Invitrogen) prior to use. The k-VitroHES™-medium may be used for up to one week if stored at +4° C. When stored at −20° C. for up to two months, no sign of reduced bioreactivity could be detected upon usage.

### Feeder-free Example 2

### Transferring of hBS Cell Lines to Feeder Free Growth Conditions

Initial hBS cell lines were maintained on Mitomycin C treated mouse feeders in 10-50 passages and cultured in VitroHES™-medium supplemented with 4 ng/ml of human basic fibroblast growth factor (bFGF).

Two different techniques were evaluated for transferring of the hBS cells from feeder culture to Matrigel™ coated plates, one with mechanical dissociation and one with collagenase treatment. The hBS cells were cut in square pieces, which represented the middle of the colony, by using a stem cell cutting tool (Swemed Lab AB, Billdal, Sweden), and carefully detached and transferred the cells to HBSS solution. The stem cell tool is a sterile sharpened glass capillary, with a 25 degree angle and a 200 or 300 micrometer lumen, designed for cutting, manipulation, and transfer of hBS colonies, or parts of hBS colonies. It is produced by Swemed Lab International AB, Bilidal, Sweden.

Enzymatic Treatment with Collagenase (for Comparison)

After washing in HBSS the cell clusters were transferred to a Collagenase IV solution (200 U/ml; Sigma) for enzymatic dissociation. The cells were incubated for 30 minutes at 37° C. and 5% CO₂. During the incubation period, repeated mechanical dissociations with a pipette were performed and the dissociation process monitored in an inverted microscope. After the incubation period the cell suspension was pelleted (400 G for 5 minutes) and washed once in KnockOut™ D-MEM (Gib coBRL/Invitrogen) before being resuspended in k-VitroHES medium.

Mechanical Dissociation According to the Invention

After washing in HBSS the cell clusters were carefully dissociated mechanically by using a 1-ml automatic pipette. The dissociation process was completed when the size of the cell clusters represented approximately 1/10-1/20 of the original colonies (average of 20 000 cells/original colony) corresponding to the size of cell aggregates generated by Collagenase IV treatment, as described above After washing in HBSS the colonies were transferred to collagenase IV solution (200 U/ml) to start the enzyme dissociation. For the two different techniques, the cells were seeded into four wells each and incubated at 37° C. in 5% CO₂. Each experiment was repeated four times, with the same amount of cells seeded each time. After two and six days the colony size and number was calculated.

Results of Feeder-free Example 1 and 2

To optimize the transferring of the hBS cultures from feeder to feeder-free conditions, two different techniques were evaluated; one with mechanical dissociation and one with enzymatic dissociation. Mechanical dissociation resulted in a more efficient attachment of cells to the Matrigel™ and a more rapid proliferation compared to the enzyme treated cultures. A significantly higher number of surviving colonies were observed two days after plating, when mechanical dissociation was compared with enzymatic dissociation (FIG. 5). The total area of all colonies generated on Matrigel™ after dissociation with the two different techniques, respectively, was compared (P<0.001). Furthermore, six days after plating the total colony area in the mechanically dissociated cultures were significantly increased compared with the enzymatically dissociated cultures (P=0.036).

### Feeder-free Example 3

### Culture and Passage of hBS Cells Cultured on Matrigel™

Four different cell lines SA 002, AS 038, SA 121 and SA 167 were used in all experiments. The cell lines were propagated on Matrigel™ for up to 35 passages and the morphological appearance and other hBS characteristics remained unaltered even after a cycle of freeze/thawing. All cultures consisted of well defined colonies of hBS cells without morphological signs of differentiation. After about 3-6 days the cells were passaged by taken away the medium and 1 ml of Collagenase IV (200 U/ml) solution was added to each well and incubated for 15-20 minutes. To facilitate cell detachment from the surface mechanical dissociation was performed followed by another 15 minutes of incubation. The cells were then washed, resuspended in k-VitroHES™ medium and seeded at a split ratio of 1:2 to 1:6 onto Matrigel™. The hBS cultures were passaged every 5 to 6 days and the medium was changed every second to third day.

Result of Feeder-free Example 3

Observations were made that during passage of the hBS cells established on Matrigel™, enzyme treatment with Collagenase IV was needed to detach the colonies from the surface. Enzymatic treatment during passage was also found to give an increased proliferation rate after seeding, compared to mechanical dissociation.

### Feeder-free Example 4

### Cryopreservation and Thawing of hBS Cells Cultured on Matrigel™

Four different cell lines SA 002, AS 038, SA 121 and SA 167 were treated with collagenase IV for 20-30 minutes to separate the cells from each other before freezing. After centrifugation the cells were transferred to freezing medium, which contains k-VitroHES™-medium containing 10% DMSO, 30% serum replacement and 4 ng/ml of bFgF, in a concentration of 1 million cells per ml freezing medium. The final cell suspension was a mixture of both single cells and cell clusters. The cryotubes (0.5-1.0 ml of cell suspension) were rapidly transferred to Nalgene freezing container for storages in −80° C. over night or at least for 2 hours before long-term storage in Liquid Nitrogen.

Thawing of the hBS Cells

k-VitroHES™-medium has to be prepared and preheated before thawing the cells by placing the cryotubes in 370 water bath until all of the cell suspension was thawed. The cell suspension was transferred to the preheated medium for 5 minutes before centrifugation (400 G in 5 minutes). Matrigel™ thin layer coated (BD) wells were rehydrated by adding 1 ml of k-VitroHES™-medium to the wells and incubate 30 minutes in 37° C. The cell pellet was resuspended in k-VitroHES™-medium and transferred to either 24- or 6-well Matrigel™ plates.

### Feeder-free Example 5

### Characterization of Feeder Free Cultured hBS Cells

All characterization experiments were performed after establishment on Matrigel™ and after a cycle of freeze/thaw.

Immunocytochemistry: The cultures were passaged as described above, seeded into 6- or 24-well Matrigel™ plates and cultured for six days before performing the immunostaining. The cultures were washed in PBS, fixed with 4% formaldehyde (HistoLab, Gothenburg, Sweden) for 15 minutes at room temperature and then washed again three times in PBS. The monoclonal primary antibodies used were directed against SSEA-1, -3 and -4 (1:200; Developmental Studies Hybridoma Bank, University of Iowa, Iowa City, Iowa), Tra-1-60, Tra-1-81 (1:200; Santa Cruz Biotechnology, Santa Cruz, Calif.), and polyclonal rabbit anti-Phospho-Histone H3 (1:150; KeLab, Upstate). The primary antibodies were incubated over night at 4° C. before being visualized using appropriate Cy3- or FITC-conjugated secondary antibodies (1:300; Jackson ImmunoResearch Laboratories, West Grove, Pa.). Cultures were also incubated with 4′-6′Diamidino-2-phenylindole (DAPI; Sigma-Aldrich Sweden AB, Stockholm, Sweden), at a final concentration of 0.5 ug/mL for 5 minutes at room temperature, to visualize all the cell nuclei. The stained cultures were rinsed and mounted using DAKO fluorescent mounting medium (Dakopatts AB, Älvsjö, Sweden) and visualized in an inverted fluorescent microscope (Nikon Eclipse TE2000-U). Alkaline phosphatase (AP) staining of the Matrigel™ cultured hBS cells was carried out according to the manufacturer's instructions using a commercially available kit (Sigma-Aldrich).

Telomerase Activity: Matrigel™ cultured hBS cells were harvested, lysed and telomerase activity analyzed by a PCR-based ELISA (Roche Diagnostics GmbH, Mannheim, Germany) according to manufacturers instructions.

Karyotyping and FISH: The Matrigel™ propagated hBS cells designated for karyotyping were incubated for 1 to 3 hours in colcemid (0.1 μg/ml, Invitrogen, Carlsbad, Calif., USA), dissociated, fixated, mounted on glass slides and the chromosomes visualized by using a modified Wrights staining (#WS-32, Sigma). Preparation of metaphase plates was performed as previously described. For the fluorescence in situ hybridization (FISH) analysis, a commercially available kit (MultiVysion™ PB Multicolour Probe Panel; Vysis, Inc., Downers Grove, Ill.) containing probes for chromosome 13, 18, 21 and the sex chromosomes (X and Y) was used according to the manufacturer's instructions. Slides were analyzed using an invert microscope equipped with appropriate filters and software (CytoVision, Applied Imaging, Santa Clara, Calif.).

Teratomas: For the teratoma formation experiment, immunodeficient SCID mice (C.B-17/IcrCrl-scidBR, Charles River Laboratories, Germany) were used. Matrigel™ propagated hBS colonies were enzymatically detached from the surface by using Collagenase IV (200 U/ml), mechanically dissociated into small cell aggregates and approximately 50 000 to 100 000 cells/organ were injected under the kidney capsule. Control animals were treated with Cryo-PBS injections or with primary brain cells from a littermate. The animals were sacrificed eight weeks after injection and the tumors were immediately fixed in a 4% solution of paraformaldehyde and paraffin embedded. For histological analysis the teratoma were sectioned to 8 μm and stained with Alcian Blue/Van Giesson.

RT-PCR Analysis of Oct-4 Expression: Total RNA was isolated from all four Matrigel™ cultured hBS cell lines by using RNeasy Mini Kit (Qiagen) according the manufacturer's instructions. The cDNA was synthesized from 1 μg of total RNA using AMV First Strand cDNA Synthesis Kit (Roche) and the PCR reaction preformed by using Platinum Taq DNA Polymerase (Invitrogen). The PCR reaction included four initial step-down cycles, with two repeated cycles for every annealing temperature, with denaturation for 15 seconds at 94° C., annealing temperature for 15 seconds at 66° to 60° C. and extension for 30 seconds at 72° C. The following cycles included 35 repeats with annealing temperature at 58° C. The forward and reverse primer sequences for Oct-4 were previously described. β-actin primers were used as internal controls (sense, 5′-TGGCACCACACCTTCTACAATGAGC-3′; antisense, 5′-GCACAGCTTCTCCTTAATGTC-ACGC-3′; 400 bp product). The PCR products were size fractioned by gel electrophoresis using a 1.5% agarose gel. Human liver was used as a positive control and water as negative control for the PCR reaction.

Results of Feeder-free Example 4 and 5

Cell lines SA 002, AS 038, SA 121 and SA 167 were frozen and thawed by using cryopreservation techniques to see if any changes in the characterization could be found. After thawing all four cell lines survived and started to grow on Matrigel™ coated plates in similar pattern

Pluripotency and maintenance of the four different hBS cell lines in feeder-free conditions was demonstrated and compared to previous results for feeder cultures of the respective cell lines. These characterizations were performed by examining the morphology, expression of undifferentiated markers, telomerase activity, karyotype, and differentiation in vivo.

Immunocytochemistry: SSEA-1 expression was negative in all feeder-free cultured hBS cell lines as opposed to staining with antibodies against SSEA-3, SSEA-4, TRA-1-60 and TRA 1-80 which show a clear positive immunoreaction as expected for pluripotent hBS cells. Further, the cells displayed high levels of AP reactivity in all four Matrigel™ propagated cell lines.

Telomerase Activity: Analysis was preformed on three of the Matrigel™ cultured hBS cell lines (AS 038, SA 121 and SA 167). The hBS cells cultured on Matrigel™ were found to have high levels of telomerase activity.

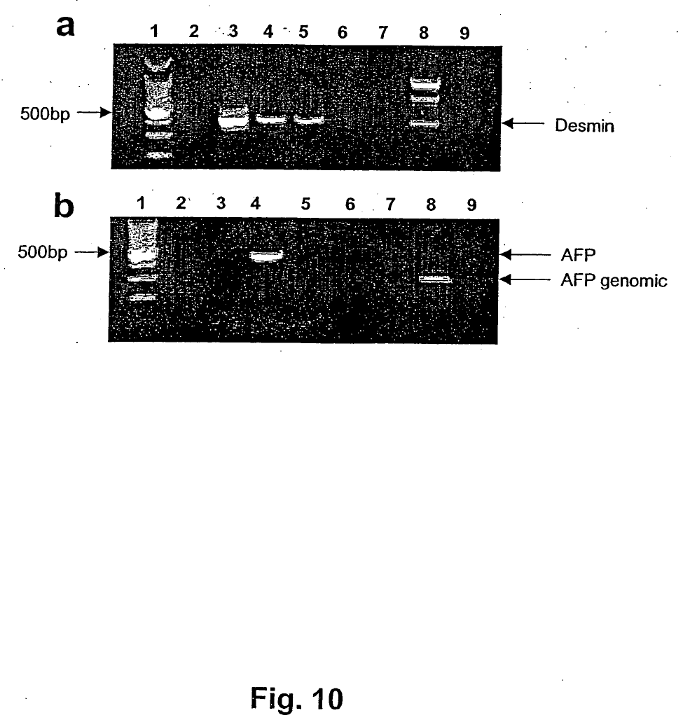

Karyotyping and FISH: Karyotype analysis was preformed on two of the Matrigel™ cultured cell lines, AS 038 and SA 121. Three of three cells from cell line AS 038 and ten of twelve cells from cell line SA 121 were found to possess normal human 46, XY karyotype (FIG. 10). The remaining two cells from the SA 121 cell line expressed an abnormal karyotype of 45, XY and 42, XY. Although, karyotypic changes seem to be normal occurring events after prolonged culturing for both feeder and feeder-free hBS cell cultures. In this study karyotypic analysis of feeder cultured hBS cells were comparable with results after Matrigel™ propagation, suggesting that the hBS cell karyotype remains normal and stable under these feeder-free conditions. FISH analysis was performed on two of the Matrigel™ propagated cell lines (SA 121 (XY) and SA 167 (XX)). Analysis was performed for chromosomes X, Y, 18, 13 and 21. For both cell lines tested at least 93% were normal. The results from the FISH analysis were comparable with results from feeder cultured hBS cell lines.

Teratoma Formation: Teratoma formation was performed for two Matrigel™ cultured hBS cell lines, SA 167 and SA 002, and the results showed that teratomas formed consisting of differentiated cells and tissue representative from all three germ layers (endoderm, mesoderm and ectoderm, providing evidence that the Matrigel™ propagated hBS cultures have retained their pluripotency.

Oct-4 Expression: Oct-4 expression was high in all four cell lines cultured on Matrigel™.

### Feeder-free Example 6

### Comparison of Mitotic Index of hBS Cells Cultured Under Feeder-free Conditions on Matrigel™Coated Plates Compared to hBS Cells Cultured on Embryonic Mouse Feeder Cells

Cell line SA 121 was cultured in parallel under feeder-free conditions on Matrigel™ coated plates and on embryonic mouse feeder cells for 3 days. The number of cells in mitosis was then quantified by nuclear immunoreactivity for phosphorylated Histone H3. The mitotic index in both cultures was calculated in order to compare the growth rate between feeder-free and feeder cultured hBS cells,

Result of Feeder-free Example 6

The mitotic index was similar in cultures grown under feeder-free (Matrigel™) compared to feeder layer conditions. The doubling time for the feeder-free cultures was roughly the same (around 35 hours) as for feeder propagated hES cells.

1. A method for obtaining neural progenitor cells, the method comprising the steps of:
i) dissociation of undifferentiated hBS cells by enzymatic and/or by mechanical treatment to obtain hBS cell aggregates or a mixture of hBS cell aggregates and single cells,
ii) seeding the hBS cells from step i) in a growth medium on a support substrate, iii) culturing the hBS cells seeded as in step ii),
iv) optionally, replacing a part of the growth medium with a growth medium,
v) passaging the obtained cells from step iii) or iv) to obtain neural progenitor cells vi) optionally, seeding the cells from step v) on a support substrate in a growth medium,
vii) optionally, repeating step v) and/or vi) at least 1 time.

2. A method according to claim 1, wherein the neural progenitor cells are produced without a step involving formation of embryoid bodies.

3. (canceled)

4. A method according to claim 1, wherein the hBS cell are propagated on feeder cells prior to step i).

5. A method according to claim 1, wherein the hBS cells are propagated in a feeder-free culture system prior to step i).

6. (canceled)

7. A method according to claim 1, wherein the hBS cells employed in step i) have at least one of the following properties:
i) exhibit proliferation capacity in an undifferentiated state for more than 12 months when grown on mitotically inactivated embryonic feeder cells or under feeder free growth conditions,
ii) exhibit normal euploid chromosomal karyotype, and
iii) maintain potential to develop into derivatives of all types of germ layers both in vitro and in vivo,
iv) exhibit at least two of the following markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2,
v) do not exhibit molecular marker SSEA-1 or other differentiation markers, and vi) retain their pluripotency and forms teratomas in vivo when injected into immunocompromised mice,
vii) are capable of differentiation.

8. A method according to claim 7, wherein the hBS cells have the properties i)-vii).

9. A method according to claim 1, wherein the dissociation of hBS cells in step i) is performed by enzymatic treatment.

10. A method according to claim 9, wherein the dissociation of hBS cells in step i) is performed by a collagenase.

11. A method according to claim 9, wherein the dissociation of hBS cells is performed by treating hBS cells with 200 U/ml collagenase at 37° C. from about 5 minutes to about 40 minutes.

12. A method according to claim 1, wherein the size of the cell aggregates obtained in step i) is from about 5 cells to about 200 cells.

13. A method according to claim 1, wherein the number of cells seeded on the support substrate is from about 40 000 cell/cm² to about 200 000 cell/cm².

14. A method according to claim 1, wherein the support substrate comprises an extracellular matrix component.

15. A method according to claim 1, wherein the support substrate comprises one or more substances selected from the group consisting of gelatin, laminin, polyornithine, fibronectin, Matrigel™, agarose, poly-L-lysine, poly-D-lysine and collagen type I.

16. A method according to claim 1, wherein the support substrate comprises gelatin.

17. A method according to claim 16, wherein the concentration of gelatin is from about 0.001% (w/v) to about 0.2% (w/v).

18. A method according to claim 1, wherein the growth medium is selected from the group consisting of hBS cell medium, VitroHES™-medium, neural cell medium, Neurobasal™ media and DMEM/F12 based medias including supplementation with B27 supplement and/or N2 supplement.

19. A method according to claim 1, wherein the growth medium is hBS cell medium or VitroHES™-medium.

20. A method according to claim 1, wherein the growth medium has been conditioned by feeder cells.

21. A method according to claim 1, wherein the growth medium employed in the different steps of the method are the same or different.

22. A method according to claim 1, wherein the growth medium comprises one or more growth factors.

23. A method according to claim 22, wherein the one or more growth factors are FGF and/or EGF.

24. A method according to claim 22, wherein the growth factor is FGF, such as, e.g. FGF-2 or hFGF.

25. A method according to claim 23, wherein the concentration of FGF and/or EGF is from about 1 ng/ml to about 40 ng/ml.

26. A method according to claim 1, wherein the culturing is preformed at 37° C., 5% CO₂ and 95% humidity.

27. A method according to claim 1, wherein the culturing in step iii) is performed for about 6 to 10 days.

28. A method according to claim 1, wherein the culturing in step iii) is performed for no longer than 8 days.

29. A method according to claim 1, wherein step iv) is included.

30. A method according to claim 29, wherein the growth medium from step ii) at least partly is replaced with fresh medium in step iv).

31. A method according to claim 30, wherein the part of the growth medium replaced in step iv) is from about 10% to about 100% v/v.

32. A method according to claim 30, wherein the fresh growth medium in step iv) may be the same or different to the growth medium from step ii).

33. A method according to claim 29, wherein step iv) is performed after at least 1 day of culture in step iii).

34. A method according to claim 1, wherein step v) includes mechanical or enzymatic treatment of the cells obtained from step iii) or, if relevant, step iv) and further culturing in a growth medium and on a support substrate.

35. A method according to claim 34, wherein the growth medium is the same or different as that used in step ii) and/or, if relevant, step iv).

36. A method according to claim 34, wherein the support medium is the same or different as that used in step ii) and/or, if relevant, step iv).

37. A method according to claim 1 including step vi).

38. A method according to claim 1 including step vii).

39. A method according to claim 1, wherein the progenitors obtained are viable after having been frozen and thawed.

40. A method according to claim 37, wherein step v) is repeated from about 1 to about 30 times.

41. A method according to claim 37, wherein step v) is repeated every 3-6 days.

42. A method according to claim 37, wherein step v) is repeated at about 60-90% confluence.

43. A method according to claim 37, wherein the cells obtained in step v) are grown as a monolayer.

44. A method according to claim 1, wherein the cells obtained have the following properties:
a) exhibit at least one of the following markers nestin, internextin, PSA-NCAM (or PS-NCAM or NCAM), Musashi-1, GAP-43, Cystatin C, Vimentin, A2B5, AC1 33, ATF5, Sox-2 and PAX-6,
b) are capable of differentiating,
c) are capable of self renewal.

45. A method according to claim 1, wherein the majority of cells obtained do not exhibit one or more markers for undifferentiated hBS cells.

46. A method according to claim 45, wherein the markers for undifferentiated hBS cells are selected from the group consisting of SSEA-3, SSEA-4, GCTM-2, Tra-1-60, Tra-1-80, Oct-4, Cripto, Rex-1 or FGF4.

47. A method according to claim 1, wherein the majority of cells obtained do not exhibit one or more markers for the endoderm germ layer, such as, e.g., alfa-fetoprotein or HNF3-α.

48. A method according to claim 1, wherein the majority of cells obtained do not exhibit one or more markers for the mesoderm germ layer, such as, e.g., cardiac troponin 1, Brachyury or Desmin.

49. A method according to claim 1, wherein the majority of cells obtained do not exhibit markers for mature neuronal cells, such as, e.g., β-III-Tubulin, MAP2, NF-L, NF-H, NF200, NF68, DoubleCortin, ChAT, DDC, GABA, DBH, NSE, synaptophysin, TH, Nurr-1, NeuN, glutamate or serotonin.

50. A method according to claim 1, wherein the majority of cells obtained do not exhibit one or more markers for glial cells, such as, e.g., GFAP, S-100, GaIC, RIP.

51. A method according to claim 45, wherein the majority of cells corresponds to 80% of more of the cells.

52. A method according to claim 45, wherein the cells are obtained from step v) or, if relevant, from step vi).

53. A method according to claim 1, wherein the cells obtained are further differentiated into at least one of the three neural cell lineages, e.g., astrocytes, oligodendrocytes and neurons.

54. A method according to claim 53, wherein the differentiated cells display the expression of at least one of the neuronal or glial cell type markers selected from the group consisting of β-III tubulin (TUJ1), NeuN, DoubleCortin, tyrosine hydroxylase, Map 2, NF-L, NH-H, NSE, DBH, GABA, synaptophysin, glutamate, serotonin, Nurr-1, NF200, NF68, GFAP, GaIC and RIP.

55. A method according to claim 53, wherein the differentiated cells display the expression of at least one of the oligodendrocyte cell type markers RIP or GaIC.

56. A method according to claim 53, wherein the differentiated cells display the expression of at least one of the astrocyte cell type markers GFAP or S-100.

57. A method for studying early neurogenesis wherein the neural progenitor cells obtained in claim 1 are used as an in vitro model.

58. A method for studying human neurodegenerative disorders wherein neural progenitor cells obtained in claim 1 are used as an in vitro model.

59. A screening method comprising utilizing neural progenitor cells obtained in claim 1.

60. An in vitro method for toxicity screening comprising utilizing neural progenitor cells obtained in claim 1.

61. An in vitro method for screening of potential drug substances comprising utilizing neural progenitor cells obtained in claim 1.

62. A method for the identification of potential drug substances comprising utilizing neural progenitor cells obtained in claim 1.

63. A medicine comprising an effective concentration of neural progenitor cells obtained in claim 1.

64. A medicament for the prevention and/or treatment of pathologies or diseases caused by tissue degeneration comprising an effective concentration of neural progenitor cells obtained in claim 1.

65. A medicament according to claim 64 wherein the tissue degeneration is degeneration of neural tissue.

66. A medicament for the prevention or treatment of pathologies and/or diseases in the nervous system comprising an effective concentration of neural progenitor cells obtained in claim 1.

67. A medicament according to claim 66, wherein the pathologies and/or diseases are selected from the group consisting of multiple schlerosis, spinal chord injury, encephalopathies, Parkinson's disease, Alzheimer's disease, Huntingdon's disease, stroke, traumatic brain injuries, hypoxia induced brain injuries, ischemia induced brain injuries, hypoglycemic brain injuries, degenerative disorders of the nervous system, brain tumours and neuropathies in the peripheral nervous system.

68. A medicine according to claim 63 which comprises neural progenitor cells dispersed in a pharmaceutically acceptable medium.

69. A medicine according to claim 68, wherein the medium is an aqueous medium.

70. A medicine according to claim 68 further comprising one or more additives selected from the group consisting of pH adjusting agents, stabilizers, preservatives, osmotic pressure adjusting agents, and physiologically acceptable salts.

71. A medicine according to claim 68 further comprising one or more agents selected from the group consisting of therapeutically active substances, prophylactically active substances, engraftment improving agents, viability improving agents, differentiation improving agent and immunosuppressive agents.

72. A composition comprising neural progenitor cells obtained by the method of claim 1.

73. A composition according to claim 72, wherein the amount of neural progenitor cells is at least 50% of the total cell population.

74. An essentially pure preparation of neural progenitor cells, obtained by the method according to claim 1, wherein the cells display the expression of at least one of the following neural progenitor cell type markers nestin, internextin, PSAN-CAM (or NCAM), Musashi-1, GAP-43, Cystatin C, Vimentin, A2B5, AC133, ATF5, HSA Sox-2 or PAX-6 and wherein the cells do not display expression of one or more markers for cell types from other germ layers.


SEQUENCE LISTING (12 sequences)

SEQ ID NO: 1 - DNA, 25 bp, Artificial Sequence
  Description of Artificial Sequence Synthetic primer
cgtgaagctggagaaggagaagctg

SEQ ID NO: 2 - DNA, 25 bp, Artificial Sequence
  Description of Artificial Sequence Synthetic primer
caagggccgcagcttacacatgttc

SEQ ID NO: 3 - DNA, 19 bp, Artificial Sequence
  Description of Artificial Sequence Synthetic primer
cggaaaaccaagacgctca

SEQ ID NO: 4 - DNA, 20 bp, Artificial Sequence
  Description of Artificial Sequence Synthetic primer
gccgttcatgtaggtctgcg

SEQ ID NO: 5 - DNA, 22 bp, Artificial Sequence
  Description of Artificial Sequence Synthetic primer
cagggacatccgggctcagtat

SEQ ID NO: 6 - DNA, 22 bp, Artificial Sequence
  Description of Artificial Sequence Synthetic primer
agcttccggtaggtggcaatct

SEQ ID NO: 7 - DNA, 22 bp, Artificial Sequence
  Description of Artificial Sequence Synthetic primer
actccagcatcgatcccacttt

SEQ ID NO: 8 - DNA, 22 bp, Artificial Sequence
  Description of Artificial Sequence Synthetic primer
agcttccggtaggtggcaatct

SEQ ID NO: 9 - DNA, 20 bp, Artificial Sequence
  Description of Artificial Sequence Synthetic primer
accacagtccatgccatcac

SEQ ID NO: 10 - DNA, 20 bp, Artificial Sequence
  Description of Artificial Sequence Synthetic primer
tccaccaccctgttgctgta

SEQ ID NO: 11 - DNA, 25 bp, Artificial Sequence
  Description of Artificial Sequence Synthetic primer
tggcaccacaccttctacaatgagc

SEQ ID NO: 12 - DNA, 25 bp, Artificial Sequence
  Description of Artificial Sequence Synthetic primer
gcacagcttctccttaatgtcacgc